SEQUENCE LISTING (15 sequences)

SEQ ID NO: 1 - DNA, 1500 bp, Influenza A virus
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaacgagcaggggagtgggtacgctgcagacaaagaatccactcaaaaggcaatagatggagtcaccaataaggtcaactcgattattgacaaaatgaacactcagtttgaggccgttggaagggaatttaacaacttagaaaggagaatagagaatttaaacaagaagatggaagacgggttcctagatgtctggacttataatgctgaacttctagttctcatggaaaacgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaac

SEQ ID NO: 2 - DNA, 1100 bp, Influenza A virus
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttg

SEQ ID NO: 3 - DNA, 1061 bp, Influenza A virus
gttaaaagtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttg

SEQ ID NO: 4 - protein, 337 aa, Influenza A virus
KSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGA

SEQ ID NO: 5 - protein, 149 aa, Influenza A virus
IAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRN

SEQ ID NO: 6 - protein, 41 aa, Influenza A virus
LLSRINHFEKIQIIPKSSWSSHEASLGVSSACPYQGKSSFF

SEQ ID NO: 7 - protein, 111 aa, Influenza A virus
LLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIA

SEQ ID NO: 8 - protein, 51 aa, Influenza A virus
YNNTNQEDLLVLWGIHHPNDAAEQTKLYQNPTTYISVGTSTLNQRLVPRIA

SEQ ID NO: 9 - DNA, 12 bp, Artificial Sequence
  Description of Artificial Sequence Synthetic primer
agcaaaagcagg

SEQ ID NO: 10 - protein, 351 aa, Influenza A virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGA

SEQ ID NO: 11 - DNA, 1542 bp, Influenza A virus
agcaaaagcagggtagataatcactcactgagtgacatcaacatcatggcgtctcaaggcaccaaacgatcttatgaacagatggaaactggtggagaacgccagaatgccactgagatcagggcatccgttggaagaatggttggtggaattgggaggttttacatacagatgtgcactgaactcaaactcagcgaccaggaaggaaggttgatccagaacagtataacaatagagagaatggttctctctgcatttgatgaaaggaggaacaggtacctagaggaacatcccagtgcggggaaggacccgaagaagaccggaggtccaatctaccgaagaagagacgggaaatgggtgagagagctgattctgtatgacaaagaggagataaggagaatttggcgccaagcgaacaatggagaagacgcaactgctggtctcactcacatgatgatctggcattccaacctaaatgatgccacataccagagaacaagagccctcgtgcggactggaatggaccccagaatgtgctctctgatgcaaggatcaaccctcccgaggagatctggagctgctggtgcagcaataaagggagtcgggacgatggtaatggaactaattcggatgataaagcgaggcattaatgaccggaacttctggagaggcgagaatggacgaagaacaaggattgcatatgagagaatgtgcaacatcctcaaagggaaatttcaaacggcagcacaaaaagcaatgatggatcaggtgcgagaaagcagaaatcctgggaatgctgaaattgaagatctcatttttctggcacggtctgcactcatcctgagaggatccgtagcccataagtcctgcttgcctgcttgtgtgtacggactcgctgtggccagtggatatgattttgagagggaagggtactctctggttgggatagatcctttccgtctgcttcagaacagtcaggtcttcagtctcattagaccaaaagagaattcagcacataaaagtcaattggtatggatggcatgccattctgcagcatttgaggacctgagagtctcaagtttcattagaggaacaagagtaatcccaagaggacaactatccactagaggagttcagattgcttcaaatgagaacgtggaagcaatggactccagcactcttgaactgagaagcagatattgggctataaggaccaggagtggaggaaacaccaaccaacagagagcatctgcaggacaaatcagtgtacagcccactttctcagtacagagaaatcttcccttcgaaagagtgaccattatggccgcgtttaaggggaataccgagggcagaacatctgacatgaggactgaaatcataagaatgatggaaagtgccagaccagaagatgtgtctttccaggggcggggagtcttcgagctctcagacgaaaaggcaacgaacccgatcgtgccttcctttgacatgagtaatgaaggatcttatttcttcggagacaatgcagaggaatatgacaattga

SEQ ID NO: 12 - DNA, 1496 bp, Influenza A virus
atggcgtctcaaggcaccaaacgatcttatgaacagatggaaactggtggggaacgccagaatgctactgagatcagggcatctgttgggagaatggttagtggcattgggaggttctacatacagatgtgcacagaactcaaactcagtgactatgaagggaggctgatccagaacagcataacaatagagagaatggtactctctgcatttgatgaaagaaggaacagatacctggaagaacaccccagtgcgggaaaggacccgaagaagactggaggtccaatttatcggaggagagacgggaaatgggtgagagagctaattctgtacgacaagaggagatcaggaggatttggcgtcaagcgaacaatggagaggacgcaactgctggtcttacccacctgatgatatggcattccaatctaaatgatgccacatatcagagaacgagagctctagtgcgtactggaatggacccaaggatgtgctctctgatgcaagggtcaactctcccgaggagatctggagctgccggtgcagcagtaaagggggtagggacaatggtgatggagctgattcggatgataaaacgagggatcaacgacgccaatttctggagaggcgaaaatggaagaagaacaaggattgcatatgagagaatgtgcaacatccttaaagggaaattccaaacagcagcacaaagagcaatgatggatcaagtgcgagagagcagaaatcctgggaatgctgaaattgaagatctcatttttctggcacggtctgcactcatcctgagaggatcagtggcccataagtcctgcttgcctgcttgtgtgtacggacttgcagtggccagtggatatgactttgagagagaagggtactctctggttggaatagatcctttccgcctgcttcaaaacagccaggtctttagtctcattagaccaaatgagaatccagcacataagagtcaattagtgtggatggcatgccactctgcagcatttgaggaccttagagtctcaagtttcatcagagggacaagagtggtcccaagaggacagctatccaccagaggggttcaaattgcttcaaatgagaacatggaggcaatggactccaacactcttgaactgagaagcagatattgggctataagaaccagaagcggaggaaacaccaaccagcagagggcatctgcaggacagatcagcgttcagcccactttctcggtacagagaaaccttcccttcgaaagagcgaccattatggcagcatttacaggaaatactgagggcagaacgtctgacatgaggactgaaatcataagaatgatggaaagtgccagaccagaagatgtgtcattccaggggcggggagtcttcgagctctcggacgaaaaggcaacgaacccgatcgtgccttcctttgacatgaataatgaaggatcttatttcttcggagacaatgcagaggagtatgacaattaa

SEQ ID NO: 13 - DNA, 759 bp, Influenza A virus
atgagtcttctaaccgaggtcgaaacgtacgttctctctatcatcccgtcaggccccctcaaagccgagatcgcgcagaaacttgaagatgtctttgcaggaaagaacaccgatctcgaggctctcatggagtggctaaagacaagaccaatcctgtcacctctgactaaagggattttgggatttgtattcacgctcaccgtgcccagtgagcgaggactgcagcgtagacgctttgtccagaatgccctaaatggaaatggagatccaaataatatggatagggcagttaagctatataagaagctgaaaagagaaataacattccatggggctaaggaggtcgcactcagctactcaaccggtgcacttgccagttgcatgggtctcatatacaacaggatgggaacggtgaccacggaagtggcttttggcctagtgtgtgccacttgtgagcagattgcagattcacagcatcggtctcacagacagatggcaactatcaccaacccactaatcagacatgagaacagaatggtgctggccagcactacagctaaggctatggagcagatggcgggatcaagtgagcaggcagcggaagccatggagatcgctaatcaggctaggcagatggtgcaggcaatgaggacaattgggactcatcctaactctagtgctggtctgagagataatcttcttgaaaatttgcaggcctaccagaaacgaatgggagtgcagatgcagcgattcaagtga

SEQ ID NO: 14 - DNA, 982 bp, Influenza A virus
atgagtcttctaaccgaggtcgaaacgtacgttctctctatcatcccgtcaggccccctcaaagccgagatcgcgcagaaacttgaagatgtctttgcaggaaagaacaccgatctcgaggctctcatggagtggctaaagacaagaccaatcctgtcacctctgactaaagggattttgggatttgtattcacgctcaccgtgcccagtgagcgaggactgcagcgtagacgctttgtccagaatgccctaaatggaaatggagatccaaataatatggatagggcagttaagctatataagaagctgaaaagagaaataacattccatggggctaaggaggtcgcactcagctactcaaccggtgcacttgccagttgcatgggtctcatatacaacaggatgggaacggtgaccacggaagtggcttttggcctagtgtgtgccacttgtgagcagattgcagattcacagcatcggtctcacagacagatggcaactatcaccaacccactaatcagacatgagaacagaatggtgctggccagcactacagctaaggctatggagcagatggcgggatcaagtgagcaggcagcggaagccatggagatcgctaatcaggctaggcagatggtgcaggcaatgaggacaattgggactcatcctaactctagtgctggtctgagagataatcttcttgaaaatttgcaggcctaccagaaacgaatgggagtgcagatgcagcgattcaagtgatcctattgttgttgccgcaaatatcattgggatcttgcacttgatattgtggattcttgatcgtcttttcttcaaatgcatttatcgtcgccttaaatacggtttgaaaagagggcctgctacggcaggggtacctgagtctatgagggaagagtaccggcaggaacagcagagtgctgtggatgttgacgatggtcattttgtcaacatagaattggagtaa

SEQ ID NO: 15 - DNA, 1350 bp, Influenza A virus
atgaatccaaatcagaagataataaccatcggatcaatctgtatggtaactggaatagttagcttaatgttacaaattgggaacatgatctcaatatgggtcagtcattcaattcacacagggaatcaacaccaatctgaaccaatcagcaatactaatttgcttactgagaaagctgtggcttcagtaaaattagcgggcaattcatctctttgccccattaacggatgggctgtatacagtaaggacaacagtataaggatcggttccaagggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggcccagacaatggggcggtggctgtattgaaatacaatggcataataacagacactatcaagagttggaggaacaacatactgagaactcaagagtctgaatgtgcatgtgtaaatggttcttgctttactgtaatgactgacggaccaagtaatggtcaggcatcacataagatcttcaaaatggaaaaagggaaagtggttaaatcagtcgaattggatgctcctaattatcactatgaggaatgctcctgttatcctgatgccggcgaaatcacatgtgtgtgcagggataattggcatggttcaaatcggccatgggtatctttcaatcaaaatttggagtatcaaataggatatatatgcagtggagttttcggagacaatccacgccccaatgatggaacaggtagttgtggtccggtgtcctctaacggggcaggtggggtaaaagggttttcatttaaatacggcaatggtgtctggatcgggagaaccaaaagcactaattccaggagcggcttcgaaatgatttgggatccaaatgggtggactgaaacggacagcagcttttcagtgaaacaagatatcgtagcaataactgattggtcaggatatagcgggagttttgtccagcatccagagctgacaggactagattgcataagaccttgtttctgggttgagttgatcagagggcggcccaaagagagcacaatttggactagtgggagcagcatatctttttgtggtgtaaatagtgacactgtgggttggtcttggccagacggtgctgagttgccattcaccattgacaagtag

(19) United States
(12) Patent Application Publication
(10) Pub. No.: US 2011/0020391 A1
(43) Pub. Date: Jan. 27, 2011

(54) RECOMBINANT MODIFIED VACCINIA VIRUS ANKARA (MVA)-BASED VACCINE FOR THE AVIAN FLU

(75) Inventors: Yasemin Süzer, Rodgau (DE); Johannes Löwer, Dreieich (DE); Gerd Sutter, Munchen (DE); A.D.M.E. Osterhaus, Antwerp (BE); G.F Rimmelzwaan, Bergschenhoek (NL); Joost Kreijtz, Uden (NL)

Correspondence Address: MILLEN, WHITE, ZELANO & BRANIGAN, P.C., 2200 CLARENDON BLVD., SUITE 1400, ARLINGTON, VA, 22201, US

(73) Assignee: PAUL-EHRLICH-INSTITUT BUNDESAMT FUR SERA UND IMPFSTOFFE, Langen (DE)

(21) Appl. No.: 12/515,716

(22) PCT Filed: Nov. 16, 2007
(86) PCT No.: PCT/EP2007/062446

§ 371 Date: Nov. 12, 2009

(30) Foreign Application Priority Data
Nov. 20, 2006 (EP) 06124336.6

(51) Int. Cl. A61K 39/285; C07H 21/04; A61P 31/12; C12N 7/01

(52) U.S. Cl. 424/199.1; 536/23.1; 435/235.1

(57) ABSTRACT

The present invention relates to the use of a recombinant modified vaccinia virus Ankara (MVA) comprising a heterologous nucleic acid for the production of a medicament in particular a vaccine, wherein the sequence of heterologous nucleic acid is from influenza A virus class H5 antigen. In a further aspect of the invention the invention relates to a recombinant modified vaccinia virus Ankara (MVA) comprising a heterologous nucleic acid, wherein (a) the heterologous nucleic acid is incorporated into a non-essential site within the genome of MVA, (b) the heterologous nucleic acid is under the control of a vaccinia virus promoter, or orthopoxvirus promoter, or poxvirus-specific promoter and, (c) the heterologous nucleic acid is selected from the group of nucleic acids encoding a gene or a part of a gene from an influenza A virus class H5.

Since the first human cases of H5N1 infections in 1997, influenza viruses of this subtype continue to cause outbreaks of fowlplague worldwide associated with an accumulating number of bird to human transmissions. As of September 14^{th}, 246 human cases were recorded of which 144 proved to be fatal (WHO disease alert 2006. Confirmed human cases of avian influenza H5N1—http://www.who.int/csr/disease/avian influenza/country/cases table 2007 10 17/en/index.html, accessed Oct. 17, 2007). In addition, the H5N1 virus infections have spread from South-East Asia to other continents of the world (WHO. 2007. Affected areas with confirmed cases of H5N1 avian influenza since 2003, status as of Aug. 10, 2007). Since these viruses not only infect avian species but also various mammalian species (Vahlenkamp, T. W., and T. C. Harder. 2006. Influenza virus infections in mammals. Berl Munch Tierarztl Wochenschr 119:123-31) including humans (Beigel, J. H., J. Farrar, A. M. Han, et al. 2005. Avian influenza A (H5N1) infection in humans. N Engl J Med 353:1374-85) there is a risk of the emerging of a new pandemic strain, either through adaptation of the avian viruses to replication in mammalian species or through the exchange of gene segments with normal epidemic influenza A viruses.

In the light of a pandemic threat caused by influenza H5N1 viruses that are transmitted from infected poultry to human in ever increasing numbers, the availability of sufficient numbers of safe and effective vaccines is considered a priority (WHO. 2007. Antigenic and genetic characteristics of H5N1 viruses and candidate H5N1 vaccine viruses developed for potential use as pre-pandemic vaccines—http://www.who.int/csr/disease/avian influenza/guidelines/summaryH5200 70403.pdf).

However, the development of such vaccines and the production of sufficient quantities of vaccine doses are not straight forward: at present the combined vaccine production capacity of all manufacturers is not sufficient to timely provide for a worldwide vaccination campaign. There is a clear need for alternative vaccine delivery systems and production technologies that could help to overcome this problem.

Since different antigenically distinct clades of H5N1 viruses have been identified recently, an ideal vaccine would also induce cross-protective immunity against these antigenic variants. Recently, conventional inactivated vaccine preparations have been evaluated, like whole inactivated virus (WIV) and split virion vaccines (Nicholson, K. G., A. E. Colegate, A. Podda, et al. 2001. Safety and antigenicity of non-adjuvanted and MF59-adjuvanted influenza A/Duck/Singapore/97 (H5N3) vaccine: a randomised trial of two potential vaccines against H5N1 influenza. Lancet 357:1937-43). These have the drawback that such a vaccine would require significant additional product characterization with regard to possible shelf life. Furthermore, implementation of such new processes by vaccine producers is not necessarily successful and tests for validation would need to be redone. In light of the danger of a pandemic these drawbacks are significant. A safe and effective adjuvant could improve the immunogenicity of conventional vaccines and may reduce the antigen-quality required to induce adequate antibody responses (dose sparing). The results herein underscore this possibility (Stimune®-adjuvanted NIBRG-14 whole virus preparation). However, at present such potent adjuvanted formulations are not considered suitable for manufacturing and applications in humans and a vaccine that does not require an adjuvant for enhancement of its immunogenicity is a favourable candidate in future vaccine development.

Vaccines based on recombinant HA expressed by baculoviruses have been tested (Treanor, J. J., B. E. Wilkinson, F. Masseoud, et al. 2001. Safety and immunogenicity of a recombinant hemagglutinin vaccine for H5 influenza in humans. Vaccine 19:1732-7). In antigenically naïve individuals these vaccines were moderately immunogenic however, and more importantly appreciable antibody responses were only induced when high doses, or a combination with an adjuvant like alum was used (Nicholson, K. G., A. E. Colegate, A. Podda, et al. 2001. Safety and antigenicity of non-adjuvanted and MF59-adjuvanted influenza A/Duck/Singapore/97 (H5N3) vaccine: a randomised trial of two potential vaccines against H5N1 influenza. Lancet 357:1937-43).

Clearly, novel vaccines are urgently needed to overcome a catastrophic shortage of vaccine in particular in the case of a H5N1 influenza pandemic. Also the drawbacks outlined above must be overcome quickly in order to avoid the consequences a pandemic would bring about.

### DESCRIPTION OF THE INVENTION

The present invention relates to the field of therapeutics more in particular the field of vaccines, more in particular the field of influenza A vaccines, more in particular H5N1 vaccines.

Influenzavirus A is a genus of a family of viruses called Orthomyxoviridae in virus classification. Influenzavirus A has only one species in it; that species is called “Influenza A virus”. Influenza A virus causes “avian influenza” (also known as bird flu, avian flu, Influenzavirus A flu, type A flu, or genus A flu). It is hosted by birds, but may infect several species of mammals. All known subtypes are endemic in birds.

The present invention solves the above outlined problems. It teaches the use of a recombinant modified vaccinia virus Ankara (MVA) comprising an isolated heterologous nucleic acid for the production of a medicament in particular a vaccine, wherein the sequence of heterologous nucleic acid is from influenza A virus class H5 antigen.

An “isolated DNA” is either (1) a DNA that contains sequence not identical to that of any naturally occurring sequence according to the invention, or (2), in the context of a DNA with a naturally-occurring sequence (e.g., a cDNA or genomic DNA), a DNA free of at least one of the genes that flank the gene containing the DNA of interest in the genome of the organism in which the gene containing the DNA of interest naturally occurs. The term therefore includes a recombinant DNA incorporated into a vector, into an autonomously replicating plasmid or virus, or into the genomic DNA of a prokaryote or eukaryote. The term also includes a separate molecule such as a cDNA where the corresponding genomic DNA has introns and therefore a different sequence; a genomic fragment that lacks at least one of the flanking genes; a fragment of cDNA or genomic DNA produced by polymerase chain reaction (PCR) and that lacks at least one of the flanking genes; a restriction fragment that lacks at least one of the flanking genes; a DNA encoding a non-naturally occurring protein such as a fusion protein, mutein, or fragment of a given protein; and a nucleic acid which is a degenerate variant of a cDNA or a naturally occurring nucleic acid. In addition, it includes a recombinant nucleotide sequence that is part of a hybrid gene, i.e., a gene encoding a non-naturally occurring fusion protein. It will be apparent from the foregoing that isolated DNA does not mean a DNA present among hundreds to millions of other DNA molecules within, for example, cDNA or genomic DNA libraries or genomic DNA restriction digests in, for example, a restriction digest reaction mixture or an electrophoretic gel slice.

Recombinant modified vaccinia virus Ankara (MVA) comprising a heterologous nucleic acid, wherein (a) the heterologous nucleic acid is incorporated into a non-essential site within the genome of MVA, (b) the heterologous nucleic acid is under the control of a vaccinia virus promoter, or orthopoxvirus promoter, or poxvirus-specific promoter and, (c) the heterologous nucleic acid is selected from the group of nucleic acids encoding a gene or a part of a gene from an influenza A virus class H5.

The term “vector” refers to a protein or a polynucleotide or a mixture thereof which is capable of being introduced or of introducing the proteins and/or nucleic acid comprised into a cell. It is preferred that the proteins encoded by the introduced polynucleotide are expressed within the cell upon introduction of the vector.

The “preferred nucleic acids for the production of a medicament in particular a vaccine”, i.e. SEQ ID NOs. 1 to 3 and 11 to 15, herein also refer to such nucleic acids, which are 95%, preferably, 98% most preferably 99% identical to those specifically listed herein. The determination of percent identity between two sequences is accomplished using the mathematical algorithm of Karlin and Altschul (1993) PNAS. 90: 5873-5877). Such an algorithm is incorporated into the BLASTN and BLASTP programs of Altschul et al. (1990) J. Mol. Biol. 215: 403-410. BLAST nucleotide searches are performed with the BLASTN program, score=100, word length=12, to obtain nucleotide sequences homologous to the EPO variant polypeptide encoding nucleic acids. BLAST protein searches are performed with the BLASTP program, score=50, wordlength=3, to obtain amino acid sequences homologous to the EPO variant polypeptide, respectively. To obtain gapped alignments for comparative purposes, Gapped BLAST is utilized as described in Altschul et al. (1997) Nucleic Acids Res. 25: 3389-3402. When utilizing BLAST and Gapped BLAST programs, the default parameters of the respective programs are used.

### DETAILED DESCRIPTION OF THE INVENTION

The inventors have found that the use of a recombinant modified vaccinia virus Ankara (MVA) comprising a heterologous nucleic acid for the production of a medicament in particular a vaccine, wherein the sequence of heterologous nucleic acid is from influenza A virus class H5 antigen solves the problems outlined above. In particular vaccination with MVA producing the HA of influenza H5N1 viruses induced potent antibody responses, even after a single vaccination, which correlated with protection against homologous and heterologous challenge infection. Most astonishingly the inventors can show that vaccination with MVA producing a specific antigen from a specific first H5N1 strain (HongKong/156/97) induces a good protection against disease upon infection with a heterologous second H5N1 (Vietnam/1194/04) strain. It is very surprising that this protection occurs in the absence of detectable antibodies against said second strain. The invention relates to the use of a recombinant modified vaccinia virus Ankara (MVA) comprising a heterologous nucleic acid for the production of a medicament in particular a vaccine, wherein the sequence of heterologous nucleic acid is from influenza A virus class H5 antigen, for the treatment in particular of the avian flu in humans but also other animals.

MVA has been originally tested in over 120,000 individuals as particularly safe vaccine against human smallpox (Mayr, A., and K. Danner. 1978. Vaccination against pox diseases under immunosuppressive conditions. Dev Biol Stand 41:225-34).

The advantages of using MVA vector vaccines include their established safety profile in humans, their efficacy upon delivery of heterologous antigens in clinical tests, and the availability of technologies for large scale vaccine production under the requirements of GMP.

The inventors have constructed different recombinant MVA viruses expressing also the hemagglutinin (HA) gene sequences of H5N1 influenza viruses A/Hongkong/156/97 (A/HK/156/97) or A/Vietnam/1194/04 (A/VN/1194/04). These viruses served as candidate vaccines in a mouse model to assess the induction of protective immunity against three different H5N1 viruses. A two-dose immunization regimen induced strong antibody responses that partially cross-reacted with heterologous H5N1 viruses; the elicited antibody responses correlated with protection against challenge infection with homologous and heterologous influenza viruses.

The vaccine according to the invention (MVA) may induce a broad immune response that protects individuals from severe clinical signs and histopathological changes in the respiratory tract even when the strains causing the infections do not fully match the vaccine antigen. The recombinant MVA vectors according to the invention have a number of properties that make them favorable vaccine candidates for use in humans.

First, recombinant MVA according to the invention can be considered as extremely safe viral vectors because of their distinct replication deficiency in mammalian cells and their well established avirulence in vivo including the safety of MVA in immune-suppressed macaques (Stittelaar, K. J., T. Kuiken, R. L. de Swart, et al. 2001. Safety of modified vaccinia virus Ankara (MVA) in immunesuppressed macaques. Vaccine 19:3700-9.) or the innocuous application of high doses of recombinant MVA to HIV-infected individuals (Harrer, E.; M. Bauerle, B. Ferstl, et al. 2005. Therapeutic vaccination of HIV-1-infected patients on HAART with a recombinant HIV-1 nef-expressing MVA: safety, immunogenicity and influence on viral load during treatment interruption. Antivir Ther 10:285-300; Goonetilleke, N., S. Moore, L. Dally, et al. 2006. Induction of multifunctional human immunodeficiency virus type 1 (HIV-1)-specific T cells capable of proliferation in healthy subjects by using a prime-boost regimen of DNA- and modified vaccinia virus Ankara-vectored vaccines expressing HIV-1 Gag coupled to CD8+ T-cell epitopes. J Virol 80:4717-28).

Second, industrial scale manufacturing of MVA vaccines appears very feasible in recognition of the efforts undertaken to develop MVA as third generation vaccine against orthopoxvirus-related biothreat (Vollmar, J., N. Arndtz, K. M. Eckl, et al. 2006. Safety and immunogenicity of IMVAMUNE, a promising candidate as a third generation smallpox vaccine. Vaccine 15; 24:2065-70).

Third, MVA vector vaccines according to the invention can deliver multiple heterologous antigens and allow for simultaneous induction of high level humoral and cellular immunity providing the possibility to develop multivalent vaccines.

Moreover, the production of MVA-based vaccines is independent of existing production capacity for conventional influenza vaccines. This way the production of MVA-based vaccines can help to reduce the envisaged shortage of vaccine doses in the time of an emerging pandemic. Another advantage is that the excellent immunogenicity of these vaccines is independent of the use of adjuvants.

A modified vaccinia virus Ankara (MVA) is a chicken cell adapted strain of vaccinia virus. Because of its avirulence found upon inoculation of animals and its striking deficiency to produce substantial amounts of new viral progeny in most cells of mammalian origin, MVA can be used under laboratory conditions of biosafety level 1. MVA serves as an efficient vector virus for expression of recombinant genes (Sutter, G., and B. Moss. 1992. Nonreplicating vaccinia vector efficiently expresses recombinant genes. PNAS 89:10847-51.) and as candidate recombinant vaccine (Moss, B., M. W. Carroll, L. S. Wyatt, et al. 1996. Host range restricted, non-replicating vaccinia virus vectors as vaccine candidates. Adv Exp Med Biol 397:7-13) with high safety profiles since MVA has been tested for pre-immunization in over 100000 humans being vaccinated against smallpox without causing notable side-effects. Several MVA vector vaccines have already entered clinical evaluation (McConkey, S. J., W. H. Reece, V. S. Moorthy, et al. 2003. Enhanced T-cell immunogenicity of plasmid DNA vaccines boosted by recombinant modified vaccinia virus Ankara in humans. Nat Med 9: 729-35; Cosma, A., R. Nagaraj, S. Bühler, et al. 2003. Therapeutic vaccination with MVA-HIV-1 nef elicits Nef-specific T-helper cell responses in chronically HIV-1 infected individuals. Vaccine 22: 21-9). Most recently, MVA is reassessed as candidate second generation vaccine against smallpox. According to the invention, the heterologous nucleic acid according to the invention is incorporated into a non-essential region of the genome of the MVA. According to the present invention, any MVA strain may be used.

WO 03/097844 A1 discloses a number of MVA strains on p. 4. One strain that may be used according to the present invention is the MVA-BN strain or a derivative thereof (WO 02/42480). Non-essential regions according to the present invention may be selected from (i) natural occurring deletion sides of the MVA genome with respect to the genome of the vaccinia virus strain Copenhagen or (ii) intergenic regions of the MVA genome. The term “intergenic region” refers preferably to those parts of the viral genome located between two adjacent genes that comprise neither coding nor regulatory sequences. However, the insertion sides for the incorporation of the heterologous nucleic acid according to the invention (non-essential region) are not restricted to these preferred insertion sides since it's within the scope of the present invention that the integration may be anywhere in the viral genome as long as it is possible to obtain recombinants that can be amplified and propagated in at least one cell culture system, such as Chicken Embryo Fibroblasts (CEF cells). Thus, a non-essential region may also be a non-essential gene or genes, the functions of which may be supplemented by the cell system used for propagation of MVA. In a preferred embodiment the heterologous nucleic acid is incorporated into a non-essential site within the genome of MVA.

In a particularly preferred embodiment the heterologous nucleic acid according to the invention is incorporated into the MVA genome at the site of deletion (III). Integration of the heterologous nucleic acid according to the invention is performed preferentially by homologous recombination of the flanking regions of a so-called transfer vector, which initially comprises the heterologous nucleic acid according to the invention prior to its integration into the MVA genome. Upon homologous integration selection of recombinant viruses is performed. One such method for selecting the recombinant viruses is expression of the host range gene K1L (see FIGS. 5 and 7).

In a preferred embodiment the heterologous nucleic acid is incorporated into the MVA genome at the site of deletion III.

According to the invention the heterologous nucleic acid is under the control of a vaccinia virus-, or orthopoxvirus-, or poxvirus-specific promoter. A number of promoters may be used for the present invention. For the expression of the heterologous nucleic acid according to the invention several promoters, such the 30K and 40K promoters (U.S. Pat. No. 5,747,324, A Strong Synthetic Early-Late Promoter; Sutter, G., L. S. Wyatt, P. L. Foley, et al. 1994. A recombinant vector derived from the host range-restricted and highly attenuated MVA strain of vaccinia virus stimulates protective immunity in mice to influenza virus. Vaccine 12:1032-1040), the P7.5 promoter (Endo, A., S. Itamura, H. Iinuma, et al. 1991. Homotypic and heterotypic protection against influenza virus infection in mice by recombinant vaccinia virus expressing the haemagglutinin or nucleoprotein of influenza virus. J Gen Virol 72: 699-703), PmH5, P11 and a promoter derived from the cowpox virus A-type inclusion (ATI) gene (Li, Y., R. L. Hall, S. L. Yan, R. W. Moyer. 1998. High-level expression of Amsacta moorei entomopoxvirus spheroidin depends on sequences within the gene. J Gen Virol 79:613-22). All of these promoters may be used according to the invention. In one embodiment it is desired that the heterologous nucleic acid is expressed in high amounts. WO 03/097844 A1 discloses such a promoter P7.5, PmH5 and P11 are preferred.

According to the invention the sequence of the heterologous nucleic acid is selected from the group of H5N1, H5N3, H5N2, H5N7, H7N1, H7N7, H7N3 and H9N2 sequences. Preferably the sequence is from H5N1.

If the heterologous nucleic acid is from H5N1 it is preferably selected from the group of strains termed, A/Vietnam/1203/04, A/Vietnam/1194/04, A/Vietnam/3046/04, A/Hongkong/156/97, A/HongKong/212/03, A/HongKong/213/03, A/Indonesia/5/05, A/Ck/Indonesia/BL03/03, A/Grey heron/HK/793.1/02, A/Black Headed Gull/HK/12.1/03, A/Dk/Vietnam/11/04, A/Ck/Vietnam/33/04, A/Ck/Vietnam/C-58/04, A/Dk/TH/D4AT/04, A/Gs/TH/G7CS/04, A/Dk/FJ/1734/05, A/Ck/GX/463/06 and A/Ck/HK/947/06.

It is preferred that the heterologous nucleic acid of H5N1 is selected from strain A/Vietnam/1194/04, A/Indonesia/5/2005, A/Bar headed goose/Qinghai/1A/2005, A/Whooper swan/Mongolia/244/2005, A/turkey/Turkey/1/2005, and A/Anhui/1/2005.

It is most preferred that the heterologous nucleic acid of H5N1 is that of strain A/Vietnam/1194/04.

It is preferred that the sequence of the heterologous nucleic acid encompasses the sequence or part of the sequence of the hemagglutinin gene. In a preferred embodiment the sequence of the heterologous nucleic acid encompasses the sequence or part of the sequence of the hemagglutinin gene from nucleotide 1 to 1500. In a more preferred embodiment from nucleotide 1 to 1100 and in a most preferred embodiment from nucleotide 40 to 1100. In a most preferred embodiment the heterologous nucleic acid encompasses the sequence or part of the sequence of the hemagglutinin gene from A/Vietnam/1194/04 selected from SEQ ID NO. 1, SEQ ID NO. 2 and SEQ ID NO. 3. SEQ ID NO. 3 is most preferred (see table 1).

TABLE 1

| | |
|---|---|
| Nucleotides 1 to | 5′- |
| 1500 of the | atggagaaaatagtgcttctttttgcaatagtcagtcttgttaaaagtgatcagatttgcattggttaccatgc |
| hemagglutinin gene | aaacaactcgacagagcaggttgacacaataatggaaaagaacgttactgttacacatgcccaagac |
| from H5N1 from | atactggaaaagacacacaatgggaagctctgcgatctagatggagtgaagcctctaattttgagagat |
| A/Vietnam/1194/04 | tgtagtgtagctggatggctcctcggaaacccaatgtgtgacgaattcatcaatgtgccggaatggtctta |
| (SEQ ID NO. 1) | catagtggagaaggccaatccagtcaatgacctctgttacccaggggatttcaatgactatgaagaattg |
| | aaacacctattgagcagaataaaccattttgagaaaattcagatcatccccaaaagttcttggtccagtc |
| | atgaagcctcattgggggtgagctcagcatgtccataccagggaaagtcctcctttttcagaaatgtggta |
| | tggcttatcaaaaagaacagtacatacccaacaataaagaggagctacaataataccaaccaagaa |
| | gatcttttggtactgtgggggattcaccatcctaatgatgcggcagagcagacaaagctctatcaaaacc |
| | caaccacctatatttccgttgggacatcaacactaaaccagagattggtaccaagaatagctactagatc |
| | caaagtaaacgggcaaagtggaaggatggagttcttctggacaattttaaaaccgaatgatgcaatca |
| | acttcgagagtaatggaaatttcattgctccagaatatgcatacaaaattgtcaagaaaggggactcaa |
| | caattatgaaaagtgaattggaatatggtaactgcaacaccaagtgtcaaactccaatgggggcgata |
| | aactctagcatgccattccacaatatacaccctctcaccatcggggaatgccccaaatatgtgaaatca |
| | aacagattagtccttgcgactgggctcagaaatagccctcaaagagagagaagaagaaaaaagag |
| | aggattatttggagctatagcaggttttatagagggaggatggcagggaatggtagatggttggtatgggt |
| | accaccatagcaacgagcaggggagtgggtacgctgcagacaaagaatccactcaaaaggcaata |
| | gatggagtcaccaataaggtcaactcgattattgacaaaatgaacactcagtttgaggccgttggaagg |
| | gaatttaacaacttagaaaggagaatagagaatttaaacaagaagatggaagacgggttcctagatgt |
| | ctggacttataatgctgaacttctagttctcatggaaaacgagagaactctagactttcatgactcaaatgt |
| | caagaacctttacgacaaggtccgactacagcttagggataatgcaaaggagctgggtaacggttgttt |
| | cgagttctatcataaatgtgataatgaatgtatggaaagtgtaagaaac-3′ |
| Nucleotides 1 to | 5′- |
| 1100 of the | atggagaaaatagtgcttctttttgcaatagtcagtcttgttaaaagtgatcagatttgcattggttaccatgc |
| hemagglutinin gene | aaacaactcgacagagcaggttgacacaataatggaaaagaacgttactgttacacatgcccaagac |
| from H5N1 | atactggaaaagacacacaatgggaagctctgcgatctagatggagtgaagcctctaattttgagagat |
| (SEQ ID NO. 2) | tgtagtgtagctggatggctcctcggaaacccaatgtgtgacgaattcatcaatgtgccggaatggtctta |
| | catagtggagaaggccaatccagtcaatgacctctgttacccaggggatttcaatgactatgaagaattg |
| | aaacacctattgagcagaataaaccattttgagaaaattcagatcatccccaaaagttcttggtccagtc |
| | atgaagcctcattgggggtgagctcagcatgtccataccagggaaagtcctcctttttcagaaatgtggta |
| | tggcttatcaaaaagaacagtacatacccaacaataaagaggagctacaataataccaaccaagaa |
| | gatcttttggtactgtgggggattcaccatcctaatgatgcggcagagcagacaaagctctatcaaaacc |
| | caaccacctatatttccgttgggacatcaacactaaaccagagattggtaccaagaatagctactagatc |
| | caaagtaaacgggcaaagtggaaggatggagttcttctggacaattttaaaaccgaatgatgcaatca |
| | acttcgagagtaatggaaatttcattgctccagaatatgcatacaaaattgtcaagaaaggggactcaa |
| | caattatgaaaagtgaattggaatatggtaactgcaacaccaagtgtcaaactccaatgggggcgata |
| | aactctagcatgccattccacaatatacaccctctcaccatcggggaatgccccaaatatgtgaaatca |
| | aacagattagtccttgcgactgggctcagaaatagccctcaaagagagagaagaagaaaaaagag |
| | aggattatttggagctatagcaggttttatagagggaggatggcagggaatggtagatggttg-3′ |
| Nucleotides 40 | 5′- |
| to 1100 of the | gttaaaagtgatcagatttgcattggttaccatgcaaacaactcgacagagcaggttgacacaataatg |
| hemagglutinin | gaaaagaacgttactgttacacatgcccaagacatactggaaaagacacacaatgggaagctctgcg |
| gene from H5N1 | atctagatggagtgaagcctctaattttgagagattgtagtgtagctggatggctcctcggaaacccaatg |
| (SEQ ID NO. 3) | tgtgacgaattcatcaatgtgccggaatggtcttacatagtggagaaggccaatccagtcaatgacctct |
| | gttacccaggggatttcaatgactatgaagaattgaaacacctattgagcagaataaaccattttgagaa |
| | aattcagatcatccccaaaagttcttggtccagtcatgaagcctcattgggggtgagctcagcatgtccat |
| | accagggaaagtcctcctttttcagaaatgtggtatggcttatcaaaaagaacagtacatacccaacaat |
| | aaagaggagctacaataataccaaccaagaagatcttttggtactgtgggggattcaccatcctaatgat |
| | gcggcagagcagacaaagctctatcaaaacccaaccacctatatttccgttgggacatcaacactaaa |
| | ccagagattggtaccaagaatagctactagatccaaagtaaacgggcaaagtggaaggatggagttc |
| | ttctggacaattttaaaaccgaatgatgcaatcaacttcgagagtaatggaaatttcattgctccagaatat |
| | gcatacaaaattgtcaagaaaggggactcaacaattatgaaaagtgaattggaatatggtaactgcaa |
| | caccaagtgtcaaactccaatgggggcgataaactctagcatgccattccacaatatacaccctctcac |
| | catcggggaatgccccaaatatgtgaaatcaaacagattagtccttgcgactgggctcagaaatagcc |
| | ctcaaagagagagaagaagaaaaaagagaggattatttggagctatagcaggttttatagagggagg |
| | atggcagggaatggtagatggttg-3′ |

Hemagglutinin (HA) is an antigenic glycoprotein found on the surface of the influenza viruses (as well as many other bacteria and viruses). It is responsible for binding the virus to the cell that is being infected. The name hemagglutinin comes from the protein's ability to cause erythrocytes to clump together (Nelson D. L. and Cox M. M., 2005. Principles of Biochemistry, 4th edition, Freeman Publishers, New York).

There are at least 16 different HA antigens. These subtypes are labeled H1 through H16. The last, H16, was discovered only recently on influenza A viruses isolated from black-headed gulls from Sweden and Norway (Fouchier R A, Munster V, Wallensten A, et al. 2005. Characterization of a novel influenza A virus hemagglutinin subtype (H16) obtained from black-headed gulls. J Virol 79:2814-22.). The first three hemagglutinins, H1, H2, and H3, are found in human influenza viruses.

A single amino acid change in the avian virus strain type H5 hemagglutinin has been found in human patients that can significantly alter receptor specificity of avian H5N1 viruses, providing them with an ability to bind to receptors optimal for human influenza viruses (Gambaryan, A, A. Tuzikov, G. Pazynina, et al. 2005. Evolution of the receptor binding phenotype of influenza A (H5) viruses. Virology 344:432-8). This finding seems to explain how an H5N1 virus that normally does not infect humans can mutate and become able to efficiently infect human cells. The hemagglutinin of the H5N1 virus has been associated with the high pathogenicity of this flu virus strain, apparently due to its ease of conversion to an active form by proteolysis (Hatta, M., P. Gao, P. Halfmann, et al. 2001. Molecular Basis for High Virulence of Hong Kong H5N1 Influenza A Viruses. Science 293:1840-1842).

It is preferred that the nucleic acid encompasses HA1 and HA2 subunits. HA is a homotrimeric integral membrane glycoprotein. It is shaped like a cylinder, and is approximately 135 Å (angstroms) long. The three identical monomers that constitute HA are constructed into a central α helix coil; three spherical heads contain the sialic acid binding sites. HA monomers are synthesized as precursors that are then glycosylated and cleaved into two smaller polypeptides: the HA1 and HA2 subunits. Each HA monomer consists of a long, helical chain anchored in the membrane by HA2 and topped by a large HA1 globule. It is also preferred that the HA1 subunit is used alone.

In further embodiment the heterologous nucleic acid encompasses the sequence coding for an amino acid sequence region selected from amino acids 1-351, amino acids 352-500, amino acids 15-351, amino acids 120-160, amino acids 180-230 and amino acids 120-230. The following are most preferred amino acids 120-160, amino acids 180-230 and amino acids 120-230. The very most preferred region is 120-160. It is most preferred that the heterologous nucleic acid encodes an amino acid sequence selected from SEQ ID NO. 4, SEQ ID NO. 5, SEQ ID NO. 6, SEQ ID NO. 7 SEQ ID NO. 8 and SEQ ID NO. 10.

TABLE 2

| | |
|---|---|
| Amino acids 15 to 351 of | ksdqicigyhannsteqvdtimeknvtvthaqdilekthngklcdldgvkplilrdcsvagwllgnpm |
| the hemagglutinin gene | cdefinvpewsyivekanpvndlcypgdfndyeelkhllsrinhfekiqiipksswssheaslgvss |
| from H5N1 from A/ | acpyqgkssffrnvvwlikknstyptikrsynntnqedllvlwgihhpndaaeqtklyqnpttyisvgt |
| Vietnam/1194/04 (SEQ ID NO. 4) | stlnqrlvpriatrskvngqsgrmeffwtilkpndainfesngnfiapeyaykivkkgdstimkseley |
| | gncntkcqtpmgainssmpfhnihpltigecpkyvksnrlvlatglrnspqrerrrkkrglfga |
| Amino acids 352 to 500 of | iagfieggwqgmvdgwygyhhsneqgsgyaadkestqkaidgvtnkvnsiidkmntqfeavgr |
| the hemagglutinin gene | efnnlerrienlnkkmedgfldvwtynaellvlmenertldfhdsnvknlydkvrlqlrdnakelgngc |
| from H5N1 from | fefyhkcdnecmesvrn |
| A/Vietnam/1194/04 (SEQ | |
| ID NO. 5) | |
| Amino acids 120 to 160 of | llsrinhfekiqiipksswssheaslgvssacpyqgkssff |
| the hemagglutinin gene | |
| from H5N1 from | |
| A/Vietnam/1194/04 (SEQ | |
| ID NO. 6) | |
| Amino acids 120 to 230 of | llsrinhfekiqiipksswssheaslgvssacpyqgkssffrnvvwlikknstyptikrsynntnqedllv |
| the hemagglutinin gene | lwgihhpndaaeqtklyqnpttyisvgtstlnqrlvpria |
| from H5N1 from | |
| A/Vietnam/1194/04 (SEQ | |
| ID NO. 7) | |
| Amino acids 180 to 230 of | ynntnqedllvlwgihhpndaaeqtklyqnpttyisvgtstlnqrlvpria |
| the hemagglutinin gene | |
| from H5N1 from | |
| A/Vietnam/1194/04 (SEQ | |
| ID NO. 8) | |
| Amino acids 1 to 351 of the | mekivllfaivslvksdqicigyhannsteqvdtimeknvtvthaqdilekthngklcdldgvkplilrdc |
| hemagglutinin gene from | svagwllgnpmcdefinvpewsyivekanpvndlcypgdfndyeelkhllsrinhfekiqiipkssw |
| H5N1 from | ssheaslgvssacpyqgkssffrnvvwlikknstyptikrsynntnqedllvlwgihhpndaaeqtkl |
| A/Vietnam/1194/04 (SEQ | yqnpttyisvgtstlnqrlvpriatrskvngqsgrmeffwtilkpndainfesngnfiapeyaykivkkg |
| ID NO. 10) | dstimkseleygncntkcqtpmgainssmpfhnihpltigecpkyvksnrlvlatglrnspqrerrrk |
| | krglfga |

In particularly preferred embodiment the heterologous nucleic acid encodes two or more sequences stemming from (a) different genes or parts thereof of a single H5N1 strain and/or, (b) different H5N1 strains. It is particularly preferred to us two or more, three or more or four or more HA sequences from different H5N1 strains. Further the two or more different sequences may be from HA and another gene of the virus.

In a preferred embodiment the sequence of the heterologous nucleic acid encompasses the sequence or part of the sequence of the hemagglutinin gene from nucleotide 1 to 1500 (SEQ ID NO. 1). In a more preferred embodiment from nucleotide 1 to 1100 (SEQ ID NO. 2) and in a most preferred embodiment from nucleotide 40 to 1100 (SEQ ID NO. 3).

A preferred sequence is the neuraminidase (NA). NA codes for neuraminidase which is an antigenic glycoprotein enzyme found on the surface of the influenza viruses. It helps the release of progeny viruses from infected cells. NA is considered as possible target antigen to elicit influenza virus-specific immune responses, i.e. antibodies and T cells that may contribute to cross-protection against different virus variants or subtypes. SEQ ID NO. 15 (Neuraminidase; Vietnam/1194/2004) is particularly preferred.

Preferred sequences are matrix sequences. Matrix encoding gene segments are matrix proteins (M1 and M2) that along with the two surface proteins (hemagglutinin and neuraminidase) make up the capsid (protective coat) of the virus. M1 is a protein that binds to the viral RNA. SEQ ID NO. 13 (M1, Vietnam/1194/2004) is preferred.

M2 is a protein that uncoats the virus exposing its contents (the eight RNA segments) to the cytoplasm of the host cell. The M2 transmembrane protein is an ion channel required for efficient infection (Robert B. Couch, Orthomyxoviruses, Baron S. (ed.) in Medical Microbiology, Fourth Edition, The University of Texas Medical Branch at Galveston. 1996. ISBN 0-9631172-1-1). The amino acid substitution (Ser31Asn) in M2 some H5N1 genotypes is associated with amantadine resistance (Guan, Y., L. L. Poon, C. Y. Cheung, et al. 2004. H5N1 influenza: A protean pandemic threat PNAS 108:8156-8161; Lee, C.-W., D. L. Suarez, T. M. Tumpey, et al. 2005. Characterization of Highly Pathogenic H5N1 Avian Influenza A Viruses Isolated from South Korea. J Virol, 79:3692-3702, Also, Pandemic Influenza Center for Infectious Disease Research & Policy Academic Health Center—University of Minnesota). SEQ ID NO. 14 (Vietnam/1194/2004; M2) is preferred. M1 and M2 are considered as possible target antigens to elicit influenza virus-specific immune responses, i.e. antibodies and T cells that may contribute to cross-protection against different influenza virus variants or subtypes.

Thus, in one embodiment two or more HA antigens are used. In one embodiment one or more HA antigens is combined with NA, M1, or M2. In a preferred embodiment M1, M2, NA, NP and HA are combined. In a very preferred embodiment these are encoded by SEQ ID NO: 11 (NP; HongKong/156/97), 12 (NP; Vietnam/1194/04), 13 (M1; Vietnam/1194/04), 14 (M2; Vietnam/1194/04) and/or SEQ ID NO. 15 (NA; Vietnam/1194/04).

In a particularly preferred embodiment the two or more sequences encode hemagglutinin or a part of hemagglutinin and stem from, A/Vietnam/1203/04, A/Vietnam/1194/04, Vietnam/3046/04, A/Bar headed goose/Qinghai/1A/2005, A/Whooper swan/Mongolia/244/2005, A/turkey/Turkey/1/2005, A/Anhui/1/2005, and/or A/IND/5/05.

| | |
|---|---|
| SEQ ID NO. 12 | atggcgtctcaaggcaccaaacgatcttatgaacagatggaaactggtggggaacgccagaatg |
| (Nucleotides 1 to 1496 of | ctactgagatcagggcatctgttgggagaatggttagtggcattgggaggttctacatacagatgtgc |
| NP from H5N1 strain | acagaactcaaactcagtgactatgaagggaggctgatccagaacagcataacaatagagaga |
| Vietnam 1194/04) | atggtactctctgcatttgatgaaagaaggaacagatacctggaagaacaccccagtgcgggaa |
| | aggacccgaagaagactggaggtccaatttatcggaggagagacgggaaatgggtgagagag |
| | ctaattctgtacgacaagaggagatcaggaggatttggcgtcaagcgaacaatggagaggacgc |
| | aactgctggtcttacccacctgatgatatggcattccaatctaaatgatgccacatatcagagaacg |
| | agagctctagtgcgtactggaatggacccaaggatgtgctctctgatgcaagggtcaactctcccg |
| | aggagatctggagctgccggtgcagcagtaaagggggtagggacaatggtgatggagctgattc |
| | ggatgataaaacgagggatcaacgacgccaatttctggagaggcgaaaatggaagaagaaca |
| | aggattgcatatgagagaatgtgcaacatccttaaagggaaattccaaacagcagcacaaagag |
| | caatgatggatcaagtgcgagagagcagaaatcctgggaatgctgaaattgaagatctcatttttct |
| | ggcacggtctgcactcatcctgagaggatcagtggcccataagtcctgcttgcctgcttgtgtgtacg |
| | gacttgcagtggccagtggatatgactttgagagagaagggtactctctggttggaatagatcctttc |
| | cgcctgcttcaaaacagccaggtctttagtctcattagaccaaatgagaatccagcacataagagt |
| | caattagtgtggatggcatgccactctgcagcatttgaggaccttagagtctcaagtttcatcagagg |
| | gacaagagtggtcccaagaggacagctatccaccagaggggttcaaattgcttcaaatgagaac |
| | atggaggcaatggactccaacactcttgaactgagaagcagatattgggctataagaaccagaa |
| | gcggaggaaacaccaaccagcagagggcatctgcaggacagatcagcgttcagcccactttctc |
| | ggtacagagaaaccttcccttcgaaagagcgaccattatggcagcatttacaggaaatactgagg |
| | gcagaacgtctgacatgaggactgaaatcataagaatgatggaaagtgccagaccagaagatgt |
| | gtcattccaggggcggggagtcttcgagctctcggacgaaaaggcaacgaacccgatcgtgcctt |
| | cctttgacatgaataatgaaggatcttatttcttcggagacaatgcagaggagtatgacaattaa |
| Nucleotides 1 to 759 of the | atgagtcttctaaccgaggtcgaaacgtacgttctctctatcatcccgtcaggccccctcaaagccg |
| matrix gene M1 of H5N1 | agatcgcgcagaaacttgaagatgtctttgcaggaaagaacaccgatctcgaggctctcatggagt |
| of Vietnam/1194/04 | ggctaaagacaagaccaatcctgtcacctctgactaaagggattttgggatttgtattcacgctcacc |
| (SEQ ID NO. 13) | gtgcccagtgagcgaggactgcagcgtagacgctttgtccagaatgccctaaatggaaatggaga |
| | tccaaataatatggatagggcagttaagctatataagaagctgaaaagagaaataacattccatg |
| | gggctaaggaggtcgcactcagctactcaaccggtgcacttgccagttgcatgggtctcatataca |
| | acaggatgggaacggtgaccacggaagtggcttttggcctagtgtgtgccacttgtgagcagattg |
| | cagattcacagcatcggtctcacagacagatggcaactatcaccaacccactaatcagacatgag |
| | aacagaatggtgctggccagcactacagctaaggctatggagcagatggcgggatcaagtgag |
| | caggcagcggaagccatggagatcgctaatcaggctaggcagatggtgcaggcaatgaggac |
| | aattgggactcatcctaactctagtgctggtctgagagataatcttcttgaaaatttgcaggcctacca |
| | gaaacgaatgggagtgcagatgcagcgattcaagtga |
| Nucleotides 1 to 982 of the | atgagtcttctaaccgaggtcgaaacgtacgttctctctatcatcccgtcaggccccctcaaagccg |
| membrane ion channel | agatcgcgcagaaacttgaagatgtctttgcaggaaagaacaccgatctcgaggctctcatggagt |
| gene M2 of H5N1 of | ggctaaagacaagaccaatcctgtcacctctgactaaagggattttgggatttgtattcacgctcacc |
| Vietnam/1194/04 | gtgcccagtgagcgaggactgcagcgtagacgctttgtccagaatgccctaaatggaaatggaga |
| (SEQ ID NO. 14) | tccaaataatatggatagggcagttaagctatataagaagctgaaaagagaaataacattccatg |
| | gggctaaggaggtcgcactcagctactcaaccggtgcacttgccagttgcatgggtctcatataca |
| | acaggatgggaacggtgaccacggaagtggcttttggcctagtgtgtgccacttgtgagcagattg |
| | cagattcacagcatcggtctcacagacagatggcaactatcaccaacccactaatcagacatgag |
| | aacagaatggtgctggccagcactacagctaaggctatggagcagatggcgggatcaagtgag |
| | caggcagcggaagccatggagatcgctaatcaggctaggcagatggtgcaggcaatgaggac |
| | aattgggactcatcctaactctagtgctggtctgagagataatcttcttgaaaatttgcaggcctacca |
| | gaaacgaatgggagtgcagatgcagcgattcaagtgatcctattgttgttgccgcaaatatcattgg |
| | gatcttgcacttgatattgtggattcttgatcgtcttttcttcaaatgcatttatcgtcgccttaaatacggttt |
| | gaaaagagggcctgctacggcaggggtacctgagtctatgagggaagagtaccggcaggaac |
| | agcagagtgctgtggatgttgacgatggtcattttgtcaacatagaattggagtaa |
| Nucleotides 1 to 1350 of | atgaatccaaatcagaagataataaccatcggatcaatctgtatggtaactggaatagttagcttaat |
| the neuraminidase gene of | gttacaaattgggaacatgatctcaatatgggtcagtcattcaattcacacagggaatcaacaccaa |
| H5N1 of Vietnam/1194/04 | tctgaaccaatcagcaatactaatttgcttactgagaaagctgtggcttcagtaaaattagcgggca |
| (SEQ ID NO. 15) | attcatctctttgccccattaacggatgggctgtatacagtaaggacaacagtataaggatcggttcc |
| | aagggggatgtgtttgttataagagagccgttcatctcatgctcccacttggaatgcagaactttcttttt |
| | gactcagggagccttgctgaatgacaagcactccaatgggactgtcaaagacagaagccctcac |
| | agaacattaatgagttgtcctgtgggtgaggctccctccccatataactcaaggtttgagtctgttgctt |
| | ggtcagcaagtgcttgccatgatggcaccagttggttgacaattggaatttctggcccagacaatgg |
| | ggcggtggctgtattgaaatacaatggcataataacagacactatcaagagttggaggaacaaca |
| | tactgagaactcaagagtctgaatgtgcatgtgtaaatggttcttgctttactgtaatgactgacggac |
| | caagtaatggtcaggcatcacataagatcttcaaaatggaaaaagggaaagtggttaaatcagtc |
| | gaattggatgctcctaattatcactatgaggaatgctcctgttatcctgatgccggcgaaatcacatgt |
| | gtgtgcagggataattggcatggttcaaatcggccatgggtatctttcaatcaaaatttggagtatcaa |
| | ataggatatatatgcagtggagttttcggagacaatccacgccccaatgatggaacaggtagttgtg |
| | gtccggtgtcctctaacggggcaggtggggtaaaagggttttcatttaaatacggcaatggtgtctgg |
| | atcgggagaaccaaaagcactaattccaggagcggcttcgaaatgatttgggatccaaatgggtg |
| | gactgaaacggacagcagcttttcagtgaaacaagatatcgtagcaataactgattggtcaggata |
| | tagcgggagttttgtccagcatccagagctgacaggactagattgcataagaccttgtttctgggttg |
| | agttgatcagagggcggcccaaagagagcacaatttggactagtgggagcagcatatctttttgtg |
| | gtgtaaatagtgacactgtgggttggtcttggccagacggtgctgagttgccattcaccattgacaag |
| | tag |

In a further aspect of the invention, the invention relates to a recombinant modified vaccinia virus Ankara (MVA) comprising a heterologous nucleic acid, wherein (a) the heterologous nucleic acid is incorporated into a non-essential site within the genome of MVA, (b) the heterologous nucleic acid is under the control of a vaccinia virus promoter, or orthopoxvirus promoter, or poxvirus-specific promoter and, (c) the heterologous nucleic acid is selected from the group of nucleic acids encoding a gene or a part of a gene from an influenza A virus class H5. A modified vaccinia virus Ankara (MVA) is a chicken cell adapted strain of vaccinia virus. Because of its avirulence found upon inoculation of animals and its striking deficiency to produce substantial amounts of new viral progeny in most cells of mammalian origin, MVA can be used under laboratory conditions of biosafety level 1. MVA serves as an efficient vector virus for expression of recombinant genes (Sutter, G., and B. Moss. 1992. Nonreplicating vaccinia vector efficiently expresses recombinant genes. PNAS 89:10847-51.) and as candidate recombinant vaccine (Moss, B., M. W. Carroll, L. S. Wyatt, et al. 1996. Host range restricted, non-replicating vaccinia virus vectors as vaccine candidates. Vaccine Adv Exp Med Biol 397:7-13) with high safety profiles since MVA has been tested for pre-immunization in over 100000 humans being vaccinated against smallpox without causing notable side-effects. Several MVA vector vaccines have already entered clinical evaluation (McConkey, S. J., W. H. Reece, V. S. Moorthy, et al. 2003. Enhanced T-cell immunogenicity of plasmid DNA vaccines boosted by recombinant modified vaccinia virus Ankara in humans. Nat Med 9: 729-35; Cosma, A., R. Nagaraj, S. Bühler, et al. 2003. Therapeutic vaccination with MVA-HIV-1 nef elicits Nef-specific T-helper cell responses in chronically HIV-1 infected individuals. Vaccine 22: 21-9). Most recently, MVA is reassessed as candidate second generation vaccine against smallpox. According to the invention, the heterologous nucleic acid according to the invention is incorporated into a non-essential region of the genome of the MVA. According to the present invention, any MVA strain may be used.

WO 03/097844 A1 discloses a number of MVA strains on p. 4. One strain that may be used according to the present invention is the MVA-BN strain or a derivative thereof (WO 02/42480). Non-essential regions according to the present invention may be selected from (i) natural occurring deletion sides of the MVA genome with respect to the genome of the vaccinia virus strain Copenhagen or (ii) intergenic regions of the MVA genome. The term “intergenic region” refers preferably to those parts of the viral genome located between two adjacent genes that comprise neither coding nor regulatory sequences. However, the insertion sides for the incorporation of the heterologous nucleic acid according to the invention (non-essential region) are not restricted to these preferred insertion sides since it's within the scope of the present invention that the integration may be anywhere in the viral genome as long as it is possible to obtain recombinants that can be amplified and propagated in at least one cell culture system, such as chicken embryo fibroblasts (CEF cells). Thus, a non-essential region may also be a non-essential gene or genes, the functions of which may be supplemented by the cell system used for propagation of MVA.

In a preferred embodiment the heterologous nucleic acid comprises M1, M2, NA, NP and HA, wherein M1, M2 and NA are located in deletion II, NP is located in deletion VI and HA is located in deletion III.

It is most preferred that these are from the following strains Hong Kong/156/97 and Vietnam/1194/2004. In a particularly preferred embodiment these have the sequences SEQ ID NO.11, 12, 13, 14 and 15.

It is preferred that the heterologous nucleic acid is selected from the group of H5N1, H5N3, H5N2, H7N7, H3N2 and H7N3 genome sequences.

It is preferred that the heterologous nucleic acid is from H5N1 and is selected from the group of strains termed, A/Vietnam/1203/04, A/Vietnam/1194/04, A/Vietnam/3046/04, A/Hongkong/156/97, A/HongKong/212/03, A/HongKong/213/03, A/Indonesia/5/05, A/Ck/Indonesia/BL03/03, A/Grey heron/HK/793.1/02, A/Black Headed Gull/HK/12.1/03, A/Dk/Vietnam/11/04, A/Ck/Vietnam/33/04, A/Ck/Vietnam/C-58/04, A/Dk/TH/D4AT/04, A/Gs/TH/G7CS/04, A/Dk/FJ/1734/05, A/Ck/GX/463/06, A/Ck/HK/947/06.

In a further embodiment the heterologous nucleic acid is from H5N1 and is selected from the group of strains termed A/Vietnam/1194/04, A/Indonesia/5/2005, A/Bar headed goose/Qinghai/1A/2005, A/Whooper swan/Mongolia/244/2005, A/turkey/Turkey/1/2005, and A/Anhui/1/2005.

It is most preferred that the heterologous nucleic acid of H5N1 is that of strain A/Vietnam/1194/04.

In one embodiment the sequence of the heterologous nucleic acid encompasses the sequence or part of the sequence of the hemagglutinin gene (see above). In further embodiment the heterologous nucleic acid encompasses the sequence coding for an amino acid sequence region selected from amino acids 1-351, amino acids 352-500, amino acids 15-351, amino acids 120-160, amino acids 180-230 and amino acids 120-230. The following are most preferred amino acids 120-160, amino acids 180-230 and amino acids 120-230. The very most preferred region is 120-160.

Furthermore, a third H5N1-variant strain was used for challenge infection of the mice: A/IND/5/05, which was antigenically distinct from the other two viruses. The recombinant MVA-HA-HK/97 was highly immunogenic. A single immunization already induced potent antibody responses against influenza virus A/HK/156/97, which were further boosted by a second vaccination. These antibodies were not cross-reactive in HI and VN assays with A/VN/1194/04 or A/IND/5/05. MVA-HA-VN/04 was less immunogenic, but after two immunizations substantial antibody responses were observed, not only against the homologous virus but also to A/HK/156/97 and to a lesser extent to A/IND/5/05. The observed antibody reactivity pattern is similar to that observed with post infection ferret sera. Thus, this asymmetry in antibody recognition pattern observed with antibodies induced by MVA-HA vaccination fully resembled that observed with antibodies induced after infection with the original influenza viruses.

The whole virus antigen NIBRG-14 vaccine preparation was included in the experiments as a positive control and was highly immunogenic in combination with the Stimune® water-in-oil adjuvant, also known as Specol being used in research for production of antigen-specific antibodies in laboratory animals. This experimental vaccine not only induced strong antibody responses to the homologous influenza virus A/VN/1194/04 but also to the other two H5N1 strains. The HI and VN antibody titers measured against the three H5N1 strains directly correlated with protection against challenge infection.

The MVA-HA-HK/97 immunized mice were only protected against a homologous challenge infection. Vaccination prevented virus replication completely and as a result neither histopathological changes nor clinical signs were observed in these mice. Although the MVA-HA-HK/97 induced antibodies did not cross-react with influenza virus A/VN/1194/04, replication of this virus was reduced and the immunized animals were protected from clinical signs (Table 1).

In contrast, no protective effects were seen against challenge infection with influenza virus A/IND/5/05. Although it is known that MVA vaccination can induce strong CTL responses which could have contributed to protection (3), it is unknown at present whether H-2b restricted cross-reactive CTL epitopes exist on the HA molecule of influenza H5N1 viruses. Immunization with MVA-HA-VN/04 induced sterilizing immunity against the homologous strain. In addition, strong protective effects were observed against the antigenically distinct influenza viruses A/HK/156/97 and A/IND/5/05. The replication of these viruses was largely reduced in most immunized animals, which correlated with the absence of infected cells in the respiratory tract and the lack of clinical signs. The protection is most likely based on virus neutralizing HA-specific serum antibodies that transudate from the circulation into the alveolar epithelium (Ito, R., Y. A. Ozaki, T. Yoshikawa, H. Hasegawa, Y. Sato, Y. Suzuki, R. Inoue, T. Morishima, N. Kondo, T. Sata, T. Kurata, and S. Tamura. 2003. Roles of antihemagglutinin IgA and IgG antibodies in different sites of the respiratory tract of vaccinated mice in preventing lethal influenza pneumonia. Vaccine 21:2362-71).

The HA sequences as well as the other sequences need not be present in their entirety. They may be for example codon optimized in order to be, e.g. better expressed.

It is preferred for the recombinant modified vaccinia virus Ankara (MVA) according to the invention the nucleic acid encompasses HA1 and HA2 subunits.

In one embodiment the nucleic acid of the recombinant modified vaccinia virus Ankara (MVA) according to the invention, encodes two or more sequences stemming from (a) different genes or parts thereof of a single H5N1 strain and/or, (b) different H5N1 strains. Such embodiments have been outlined in detail above.

In a preferred embodiment the two or more sequences encode hemagglutinin or a part of hemagglutinin and stem from, A/Vietnam/1203/04, A/Vietnam/1194/04, A/Vietnam/3046/04, A/Hongkong/156/97, A/HongKong/212/03, A/HongKong/213/03, A/Indonesia/5/05, A/Ck/Indonesia/BL03/03, A/Grey heron/HK/793.1/02, A/Black Headed Gull/HK/12.1/03, A/Dk/Vietnam/11/04, A/Ck/Vietnam/33/04, A/Ck/Vietnam/C-58/04, A/Dk/TH/D4AT/04, A/Gs/TH/G7CS/04, A/Dk/FJ/1734/05, A/Ck/GX/463/06 or A/Ck/HK/947/06.

In one embodiment the invention relates to a nucleic acid encoding a modified vaccinia virus Ankara (MVA) according to the invention outlined above.

In a further embodiment the invention relates to a method of introducing the recombinant MVA according to the invention into a target cell comprising infection of the target cell with a recombinant MVA according to the invention.

The invention also relates to method for immunization of a living animal body including a human said method comprising administering to said living animal body including a human in need thereof a therapeutically effective amount of the MVA according to the invention.

In a further embodiment the invention relates to a method of introducing a MVA according to the invention into a target cell comprising infection of the target cell with an MVA according to the invention.

Viruses are propagated and titrated following standard methodology. To generate vaccine preparations, viruses may routinely be purified by ultracentrifugation through sucrose and reconstituted in, e.g. 1 mM Tris/HCl pH 9.0. Rabbit kidney (RK13, ATCC CCL-37™) cells may be grown in minimal essential medium (MEM) supplemented with 10% fetal calf serum (FCS) and maintained at 37° C. and 5% CO₂. CEF cells may be grown in 6 well tissue culture plates and infected with the multiplicity of 20 infectious units MVA.

To determine low or high multiplicity growth profiles, confluent CEF monolayers (grown on 6 well plates) have to be infected with 0.05 infectious units (IU) or 10 IU MVA or MVA according to the invention per cell, respectively. After virus absorption for 60 min at 37° C. the inoculum has to be removed. Cells should be washed twice with RPMI 1640 and incubated with fresh RPMI 1640 medium containing 2% FCS at 37° C. and 5% CO₂. At multiple time points post infection (p.i.) infected cells can be harvested and virus can be released by freeze-thawing followed by a brief sonication. Serial dilutions of the resulting lysates have to be plated on confluent CEF monolayers grown in 6 well plates as replicates of two and after a 48 hrs incubation period monolayers may be briefly fixed in ice-cold acetone:methanol (1:1) and cells may be incubated for 60 min with polyclonal rabbit anti-vaccinia antibody (IgG fraction, Biogenesis Ltd., Pool, England), followed by an incubation for 45 min with horseradish-peroxidase-conjugated polyclonal goat anti-rabbit antibody (Dianova, Hamburg). After washing with PBS, antibody-labeled cells may be developed using an o-Dianisidine (Sigma, Taufkirchen, Germany) substrate solution, foci of stained cells may be counted, and virus titers may be calculated as IU/ml.

According to one embodiment of the present invention, the invention relates to a method for immunization of a living animal body including a human, said method comprising administering to said living animal body including a human in need of a therapeutically effective amount of the MVA according to the invention. Said method according to the invention may comprise a composition which may also contain (in addition to the ingredient and the carrier) diluents, common fillers, salts, buffers, stabilizers, solubilizers and other materials well known in the art. It is necessary that these are pharmaceutically acceptable.

The term “pharmaceutically acceptable” means a non-toxic material that does not interfere with the effectiveness of the biological activity of the MVA according to the invention. The characteristics of the carrier will depend on the route of administration. The pharmaceutical composition may further contain other agents which either enhance the activity or use in treatment. Such additional factors and/or agents may be included in the pharmaceutical composition to be applied for the method for immunization according to the invention to produce a synergistic effect or to minimize side-effects. Techniques for formulation and administration of the MVA according to the invention may be found in “Remington's Pharmaceutical Sciences”, (Muck Publishing Company, Easton, Pa., latest edition).

The method for immunization according to the present invention will make use of a therapeutically effective amount of the MVA. A therapeutically effective dose further refers to that amount of the compound/ingredient sufficient to result in amelioration of symptoms, e.g. treatment, healing, prevention or amelioration of such conditions. To prepare vaccines, the MVA vaccinia virus generated according to the invention are converted into a physiologically acceptable form. This can be done based on the many years of experience in the preparation of vaccines used for vaccination against smallpox (Kaplan C. 1969. Immunization against smallpox. Br Med. Bull. 25:131-135). Typically, about 10⁶ to 10⁷ particles of the recombinant MVA are freeze-dried in 100 ml of phosphate-buffered saline (PBS) in the presence of 2% peptone and 1 human albumin in an ampoule, preferably a glass ampoule. The lyophilisate can contain extenders, such as mannitol, dextran, sugar, glycine, lactose or polyvinylpyrrolidole or other auxiliary substances (such as anti-oxidants, stabilizers, etc.) suitable for parenteral administration. The glass ampoule is then sealed and can be stored, preferably at temperatures below −20° C. for several months. For vaccination the lyophilisate can be dissolved in 0.1 to 0.2 ml of aqueous solution, preferably physiological saline, and administered parentally, for example by intradermal inoculation. The vaccine according to the invention is preferably injected intracutaneously. Slight swelling and redness, sometimes also itching, may be found at the injection side. The mode of administration, the dose and the number of administrations can be optimized by those skilled in the art in a known manner. It is expedient where appropriate to administer the vaccine several times over a lengthy period in order to obtain a high-level immune response against the foreign antigen. In a preferred embodiment the immunization may be both prophylactic and/or therapeutic.

The invention relates also to a vaccination kit with the vaccine, pre-filled syringes with the vaccine and other modes of storage and application.

In one embodiment the living animal body is a mammal, in one it is a bird. In a preferred embodiment it is a chicken or a human.

In one embodiment said immunization may be both prophylactic and/or therapeutic.

### EXAMPLES

### Vaccine Preparation

The H5N1 viruses were propagated in MDCK cells and the viral RNA was extracted from the culture supernatants using a high pure RNA isolation kit (Roche, Almere, the Netherlands). Subsequently cDNA was synthesized from the vRNA using Superscript reverse transcriptase (Invitrogen, Carlsbad, Calif., USA) and the 5′-AGCAAAAGCAGG-3′ (SEQ ID NO. 9) oligonucleotide (Eurogentec, Seraing, Belgium) as primer. Next, the HA gene sequences were amplified by polymerase chain reaction using Pfu (Pyrococcus furiosus, Stratagene, La Jolla, Calif., USA) as heat stable DNA polymerase. Primer sequences were extended with the NotI and XhoI restriction sites to facilitate directional cloning into the cloning plasmid pBluescriptSK+ (Stratagene, La Jolla, Calif., USA).

HA gene sequences were prepared from these plasmids by NotI/XhoI digestion, treated with Klenow polymerase to generate blunt ends and cloned into the PmeI site of MVA expression plasmid pIIIΔHR-PsynII to generate the MVA vector plasmids pIII-HA-HK/97 and pIII-HA-VN/04.

Upon transfection in MVA-infected cells these plasmids direct insertion of foreign genes into the site of deletion III within the MVA genome (Sutter, G., and B. Moss. 1992. Nonreplicating vaccinia vector efficiently expresses recombinant genes. PNAS 89:10847-51.) and allow transcription of the HA target genes under control of the vaccinia virus-specific promoter PsynII (Wyatt, L. S., P. L. Earl, J. Y. Liu, et al. 2004. AIDS Res Hum Retr 20:645-653). Recombinant viruses MVA-HA-HK/97 and MVA-HA-VN/04 were generated in primary chicken embryo fibroblasts (CEF) upon transfection with 1 μg plasmid DNA, infection with 0.05 infectious units/cell MVA isolate F6 (Sutter, G., and B. Moss. 1992. Nonreplicating vaccinia vector efficiently expresses recombinant genes. PNAS 89:10847-51.), and by plaque selection on RK-13 cells (Staib, C., I. Drexler, and G. Sutter. 2004. Construction and isolation of recombinant MVA. Methods Mol Biol 269:77-100). The recombinant MVA genomes were analyzed by PCR to verify HA gene insertion and genetic stability (data not shown). The production of HA antigens by the MVA vector viruses was confirmed by Western blot analysis of CEF cell lysates harvested at various time points after infection with MVA-HA-HK/97 or MVA-HA-VN/04 (data not shown). One-step and multiple-step growth analysis in CEF demonstrated close to identical replicative capacities of MVA-HA-HK/97 and MVA-HA-VN/04 in comparison to non-recombinant MVA (data not shown).

To generate vaccine preparations the viruses were amplified in CEF, purified by ultracentrifugation through sucrose and reconstituted in 1 mM Tris/HCl pH 9.0. MVA vaccines were used at a dose of 10⁸ PFU diluted in 100 μl PBS.

Whole inactivated NIBRG-14 virus, a reassortant vaccine strain based on influenza virus A/Vietnam/1194/04 made by reverse genetics was used as positive control. The lyophilised whole virus antigen was reconstituted in distilled water at a concentration of 2 μg HA/50 μl and mixed 1:1 with the adjuvant Stimune® (Cedi-Diagnostics, Lelystad, The Netherlands). Control mice were inoculated with PBS.

### Influenza Viruses

Influenza viruses A/HK/156/97, A/VN/1194/04 and A/Indonesia/5/05 (A/IND/5/05) were inoculated in the allantoic cavity of 11-day-old embryonated chicken eggs. The allantoic fluid was harvested after 3 days. Infectious virus titers were determined in Madin-Darby Canine Kidney (MDCK) cells as described previously (Rimmelzwaan, G. F., M. Baars, E. C. Claas, et al. 1998. Comparison of RNA hybridization, hemagglutination assay, titration of infectious virus and immunofluorescence as methods for monitoring influenza virus replication in vitro. J Virol Methods 4:57-66).

### Mice

Female specified pathogen free 6-8 weeks old C57BL/6J mice were purchased from Charles River (Sulzfeld, Germany). The animals (n=90) were divided in five groups of 18 mice and immunized with either PBS, MVA-HA-HK/97, MVA-HA-VN/04, wtMVA, or Stimune®-adjuvanted NIBRG-14.

Immunization was done intramuscular, 50 μl in the left hind leg and 50 μl in the right. Four weeks later, blood was drawn by orbita puncture and animals were immunized again as described above. Another four weeks later blood was drawn and each of the five vaccine groups (n=18) was divided into three sub-groups of six animals each.

The sub-groups of each vaccine group were inoculated with 10³ TCID₅₀ of influenza virus A/HK/156/97, A/VN/1194/04 or A/IND/5/05 in 50 μl PBS by the intranasal route. Six non immunized animals were used as negative controls and were inoculated with 50 μl PBS. Animals were weighed every day until day 4 after infection and then sacrificed by exsanguination.

After sacrificing the mice the following organs were resected: brain, lungs (inflated with formalin), spleen and intestines. Animals in all groups were properly age-matched at the time point of challenge infection.

The experimental protocol was approved by an independent Animal ethics committee prior to the start of the experiment. Intramuscular immunizations, intranasal infections, orbita punctures and euthanasia were carried out under anaesthesia with inhalative isoflurane. The animals were housed in filter-top cages and had access to food and water ad libitum. During the 5 days of infection with the H5N1 influenza virus, animals were placed in filter-top cages in biosafety level 3 containment facilities. One BSL-3 isolator unit was used per virus.

### Virus Titers in Organ Tissues

Organs were snap frozen on dry ice with ethanol and stored at −70° C. Organs were homogenized with a Polytron homogenizer (Kinematica AG, Littau-Lucerne, Switzerland) in transport medium (Hanks medium (MEM), Glycerol, 100 U/ml penicillin, 100 μg/ml streptomycin, polymyxin B, nystatin, gentamicin, 7.5% NaHCO3, 1M Hepes). Quintuplicate ten-fold serial dilutions of these samples were used to determine the virus titers on confluent layers of Madin-Darby Canine Kidney Cells (MDCK cells).

### Serology

After treatment with cholera filtrate and heat-inactivation at 56° C., the sera were tested for the presence of anti-HA antibodies. For this purpose a hemagglutination inhibition assay (HI) was used following a standard protocol using 1% turkey erythrocytes and four HA-units of either influenza virus A/HK/156/97, A/VN/1194/04 or A/IND/5/05 (Palmer, D. F., M. T. Coleman, W. R. Dowdle, et al. 1975. Advanced laboratory techniques for influenza diagnosis, p. 51-52. In Immunology series No. 6. United States Department of Health, Education and Welfare, Washington, D.C). For this purpose reverse genetics viruses were produced from which the basic cleavage site was removed. The use of these reverse genetics viruses was validated and titers obtained were comparable with those against the wildtype strains (data not shown). Sera were also tested for the presence of virus neutralising antibodies specific for the three influenza viruses using a micro virus neutralisation (VN) assay with 100 TCID₅₀ of the respective viruses (Frank, A. L., J. Puck, B. J. Hughes, et al. 1980. Microneutralization test for influenza A and B and parainfluenza 1 and 2 viruses that uses continuous cell lines and fresh serum enhancement. J Clin Microbiol 12:426-32). Hyperimmune serum obtained from a swan immunized twice with inactivated H5N2 influenza virus A/Duck/Potsdam/1402/86 (Intervet, Boxmeer, the Netherlands) was used as a positive control against the 3 different influenza A viruses.

### Histopathology

Formalin-inflated lungs were fixated in 10% neutral buffered formalin, embedded in paraffin, sectioned at 4 μm and stained with hematoxylin and eosin for histological evaluation. Furthermore slides were stained using an immunoperoxidase method with a monoclonal antibody (Clone HB65 IgG2a (American Type Culture Collection)) directed against the nucleoprotein of influenza A virus. A Goat-anti-mouse IgG2a HRP (Southern Biotech, Birmingham, Ala., USA) was used as the secondary antibody. The peroxidase was revealed using diaminobenzidine as a substrate, resulting in a deep red precipitate in the nuclei of influenza A infected cells and a less intense red staining of the cytoplasm. The sections were counterstained with hematoxylin.

### Statistical Analysis

Data for viral titers and antibody titers were analysed using the two-sided Student's t test and differences were considered significant at P<0.05.

### Clinical Signs

From day two post infection onwards, mice immunized with PBS or wtMVA developed clinical signs like hunched posture, rapid breathing, ruffled fur and decreased muscle strength irrespective of the influenza H5N1 virus that was used for infection. These clinical signs were not observed in mice infected with influenza virus A/HK/156/97 or A/VN/1194/04 after vaccination with MVA-HA-HK/97 or MVA-HA-VN/04. MVA-HA-VN/04 vaccination also prevented the development of clinical signs caused by infection with influenza virus A/IND/5/05. The observed protection against clinical signs correlated with reduced loss of bodyweight after infection (FIG. 1). In PBS and wtMVA immunized mice an average loss of bodyweight of 16.2% and 11.5% was observed post infection with influenza virus A/HK/156/97 (FIG. 1A) or 16.9% and 10.4% post infection with influenza virus A/VN/1194/04 (FIG. 1B), respectively. This loss of bodyweight was significantly reduced by vaccination with MVA-HA-HK/97 or MVA-HA-VN/04 (FIG. 1). Also infection with influenza virus A/IND/5/05 (FIG. 1C) caused severe loss of bodyweight in PBS-immunized control mice or wtMVA vaccinated mice (16.9% and 18.6% respectively), which was significantly reduced by vaccination with MVA-HA-VN/04 but not by vaccination with MVA-HA-HK/97.

### Virus Replication in Organs

Infectious virus titers were determined in brains, intestines, lungs and spleens on day 4 post infection with influenza viruses A/HK/156/97 (FIG. 2A), A/VN/1194/04 (FIG. 2B) or A/IND/5/05 (FIG. 2C).

After infection, the highest virus replication was observed in the lungs with average lung virus titers of 10^{7.9}, 10^{7.8} and 10^{8.9} TCID₅₀/gram tissue for unprotected PBS-inoculated mice subsequently infected with influenza viruses A/HK/156/97, A/VN/1194/04 or A/IND/5/05 respectively. Also mice vaccinated with wtMVA were not protected and similar average virus titers were found in the lungs of infected mice. In some animals in both groups of unprotected mice virus replication could be demonstrated in extra-respiratory tissues including brain, intestines and spleen (Table 1).

TABLE 1

| | Virus used for challenge infection | | | | | | | | | | | | | | |
|---|---|---|---|---|---|---|---|---|---|---|---|---|---|---|---|
| | A/HK/156/97 | | | | | A/VN/1194/04 | | | | | A/IND/5/05 | | | | |
| Vaccine | >10% | | | | | >10% | | | | | 10% | | | | |
| group | brain | intestines | lung | spleen | weight loss | brain | intestines | lung | spleen | weight loss | brain | intestines | lung | spleen | weight loss |
| wtMVA | 1/6 | 1/6 | 6/6 | 4/6 | 5/6 | 2/6 | 0/6 | 6/6 | 3/6 | 4/6 | 2/6 | 2/6 | 6/6 | 4/6 | 5/6 |
| MVA-HA- | 0/4 | 0/4 | 0/4 | 0/4 | 0/4 | 0/6 | 1/6 | 6/6 | 1/6 | 1/6 | 0/6 | 0/6 | 6/6 | 0/6 | 4/6 |
| HK/97 | | | | | | | | | | | | | | | |
| MVA-HA- | 2/6 | 0/6 | 2/6 | 0/6 | 0/6 | 0/6 | 0/6 | 0/6 | 0/6 | 0/6 | 0/6 | 0/6 | 2/6 | 0/6 | 0/6 |
| VN/04 | | | | | | | | | | | | | | | |
| NIBRG-14 | 0/6 | 0/6 | 0/5 | 0/6 | 0/6 | 0/6 | 0/6 | 0/6 | 0/6 | 0/6 | 0/6 | 0/6 | 0/6 | 0/6 | 0/6 |
| PBS | 0/6 | 0/6 | 5/5 | 3/6 | 4/5 | 3/5 | 2/5 | 5/5 | 3/6 | 5/5 | 0/6 | 3/6 | 6/6 | 4/6 | 6/6 |

Table 1 shows detection of infectious virus and weight loss observed in individual mice. Group numbers < 6 are caused by fatalities due to complications that had nothing to do with the experiment.

Vaccination with MVA-HA-HK/97 prevented replication of influenza virus A/HK/156/97 in the lungs and other organs completely whereas with MVA-HA-VN/04 vaccination a reduction of virus replication in the lungs was observed in four out of six mice.

After challenge infection with influenza virus A/VN/1194/04 it was the other way around: vaccination with MVA-HA-VN/04 prevented replication completely, whereas vaccination with MVA-HA-HK/97 only partially reduced virus replication.

The reduction was statistically significant compared to PBS-inoculated mice (p<0.05), while such a difference was not observed for wtMVA vaccinated mice.

Vaccination with MVA-HA-VN/04 also prevented replication of influenza virus A/IND/5/05 in the lungs of four out of six mice resulting in reduced average lungs titers compared to PBS and wtMVA vaccinated mice. Vaccination with MVA-HA-HK/97 did not prevent replication of influenza virus A/IND/5/05 and all six mice tested positive (see table 1 above). Vaccination with the inactivated whole virus vaccine NIBRG-14 adjuvanted with Stimune® not only prevented replication of the homologous strain influenza virus A/VN/1194/04 but also that of A/HK/156/97 and A/IND/5/05.

### Serology

Upon a single vaccination with MVA-HA-HK/97 mice developed strong antibody responses against the homologous virus strain, however antibodies did not cross-react with the influenza virus strains A/VN/1194/04 or A/IND/5/05 as measured by HI and VN tests (FIG. 3) (GMT of 1629 and 239 respectively). After the booster vaccination the homologous antibody titers in the HI- and VN-assay were 1370 and 744 GMT respectively. Again no cross-reaction was observed with the other H5N1 strains. The MVA-HA-VN/04 vaccine preparation was less immunogenic and after one vaccination only in one animal, antibodies against the homologous strain were detectable in the VN-assay. Upon a booster vaccination all animals responded and the GMT increased to 20 and 64 as measured by HI and VN assays respectively. The antibodies induced by MVA-HA-VN/04 vaccination cross-reacted with the H5N1 strain A/HK/156/97 and to a limited extent with the strain A/IND/5/05. The adjuvanted NIBRG-14 vaccine preparation, which was included in the experiments as a positive control induced robust antibody responses against the homologous A/VN/1194/04, which cross-reacted with the strain A/HK/156/97 and A/IND/5/05 both in the HI and VN assay.

### Pathological Changes and Virus Replication in the Lungs

Four days after infection with either of the three HPAI viruses the mice were sacrificed and their lungs were inflated with formalin and examined histologically and using immunohistochemistry. Inoculation of wtMVA- and PBS-immunized mice with influenza virus A/Hongkong/156/97 resulted in a multifocal infection of the bronchiolar and alveolar epithelium, combined with a mild peribroncheal infiltrate and mild necrosis of the bronchiolar wall (FIGS. 4A, E). In the lungs of MVA-HA-HK/97 and MVA-HA-VN/04 immunized mice no virus on day 4 post infection was detected and no histopathological changes were observed (FIGS. 4B, C). Influenza virus A/VN/1194/04 infection led to stronger multifocal infection of the peribronchiolar and alveolar epithelium in both PBS and wtMVA immunized mice (Figure AF, J). Furthermore these mice suffered from moderate interstitial pneumonia based on loss of bronchiolar epithelium, strong peribroncheal infiltration and alveolar damage. Infection of the alveolar epithelium of MVA-HA-HK/97 immunized mice appeared to be less, however alveolar infiltration was more pronounced (FIG. 4G). In the lungs of MVA-HA-VN/04 immunized mice no virus replication was detected and morphology was normal (FIG. 4H). Infection with influenza virus A/IND/5/05 caused widespread infection of both bronchiolar and alveolar epithelium, in wtMVA, PBS and MVA-HA-HK/97 immunized mice (FIGS. 4K, L, O). Furthermore moderate infiltrate in both the bronchioles and alveoli was seen, next to loss of bronchiolar epithelium and the presence of intra-bronchiolar cell debri. MVA-HA-VN/04 immunized mice had a small number of peribronchiolar influenza virus A/IND/5/05 infected cells combined with moderate peribronchiolar infiltrate (FIG. 4M). Mice immunized with adjuvanted NIBRG-14 had normal lungs, and no virus replication was seen after infection with either of the three influenza H5N1 viruses (FIGS. 4D, I, N).

### FIGURE CAPTIONS

FIG. 1

Weight loss of mice intranasally infected with 10³ TCID₅₀ of influenza virus A/Hongkong/156/97 (A), A/Vietnam/1194/04 (B), or A/Indonesia/5/05 (C). Mean weight loss is expressed as the percentage of the original weight before infection. (*) indicates a statistically significant difference with (p<0.05).

FIG. 2

Virus titers in organ tissue at day 4 after infection with either influenza virus A/Hongkong/156/97 (A), A/Vietnam/1194/04 (B), or A/Indonesia/5/05 (C). Results are shown for the wtMVA, MVA-HA-HK/97, MVA-HA-VN/04, Stimune®-adjuvanted NIBRG14 and PBS immunized mice. Titers were measured in: brain ( [symbol] ), intestines ( [symbol] ), lungs ( [symbol] ) and spleen ( [symbol] ) and presented as TCID50 per gram tissue (Log 10). (*) Indicates that at least one animal tested positive for the indicated organ.

FIG. 3

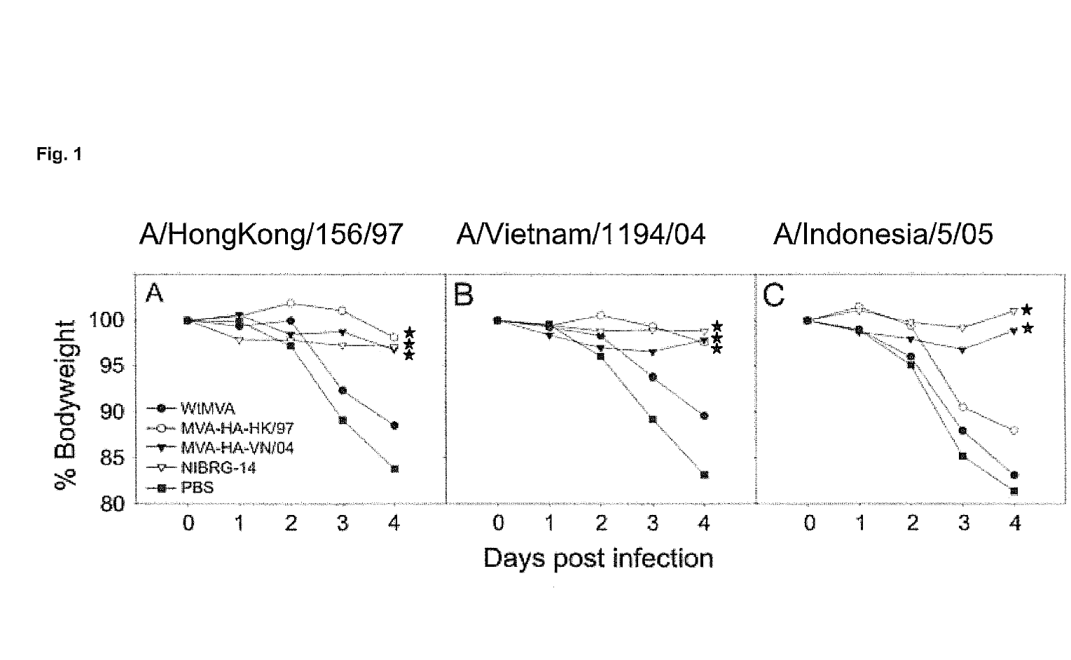

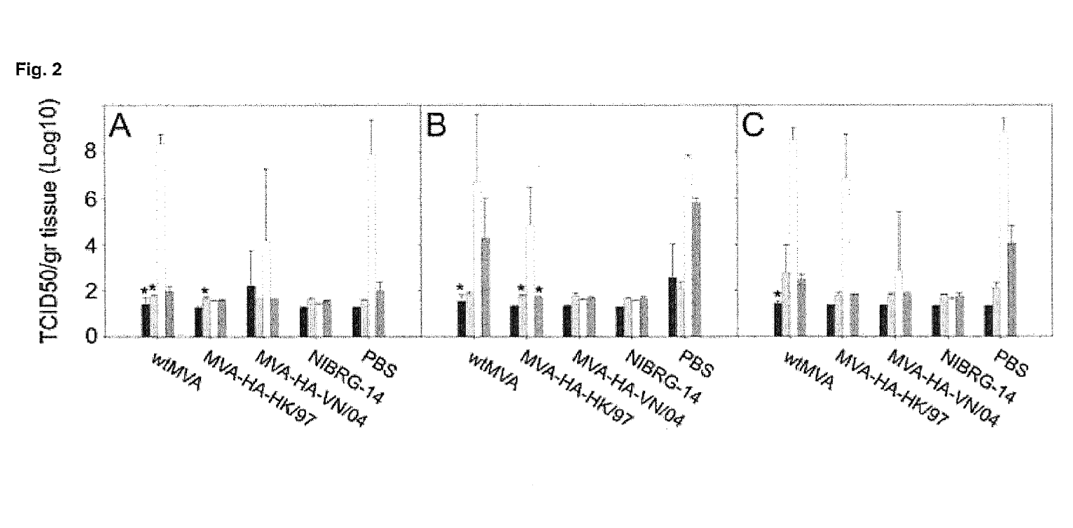

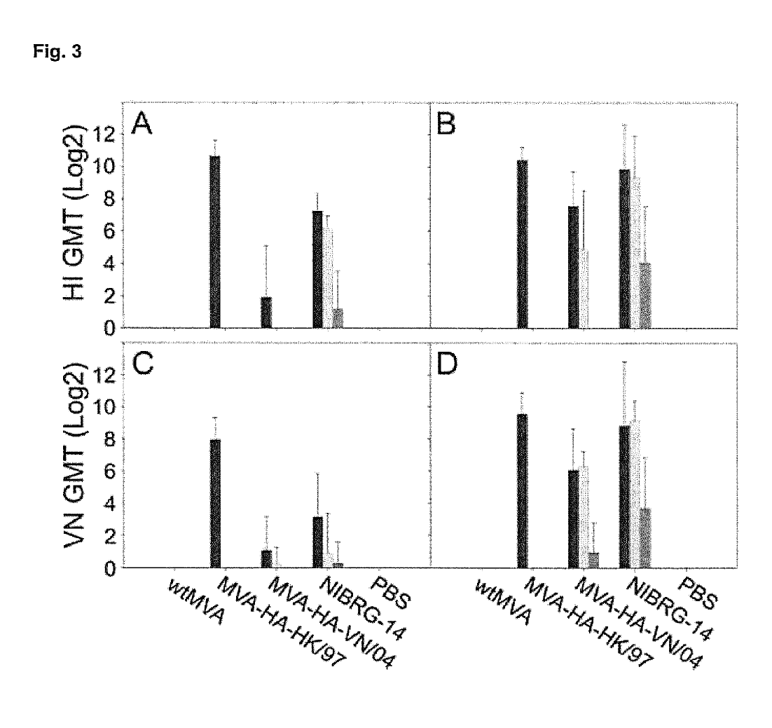

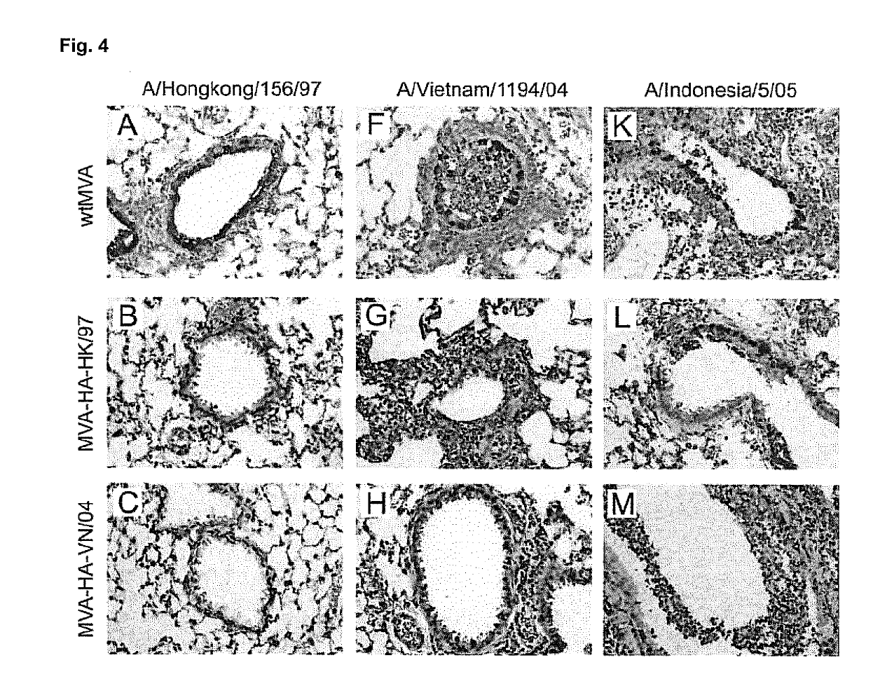

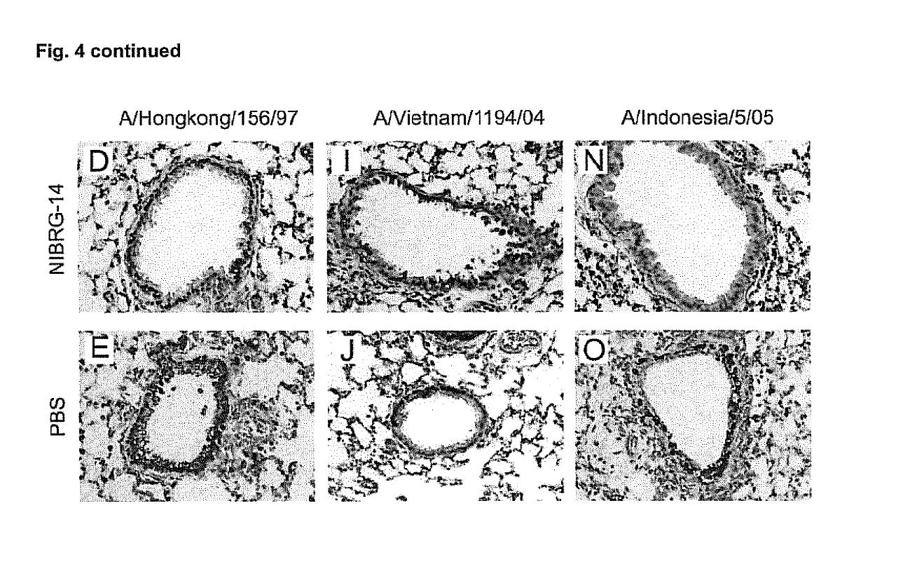

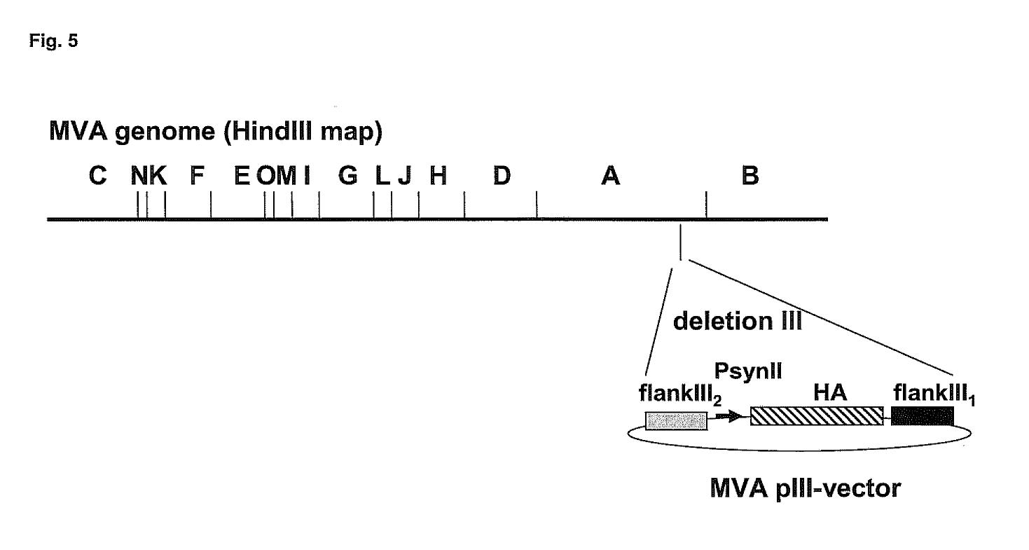

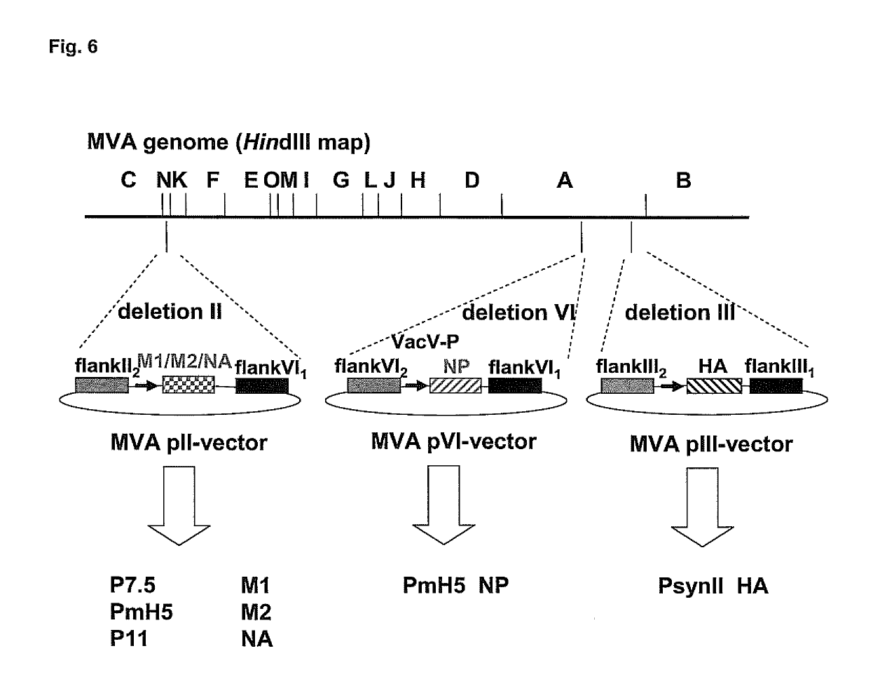

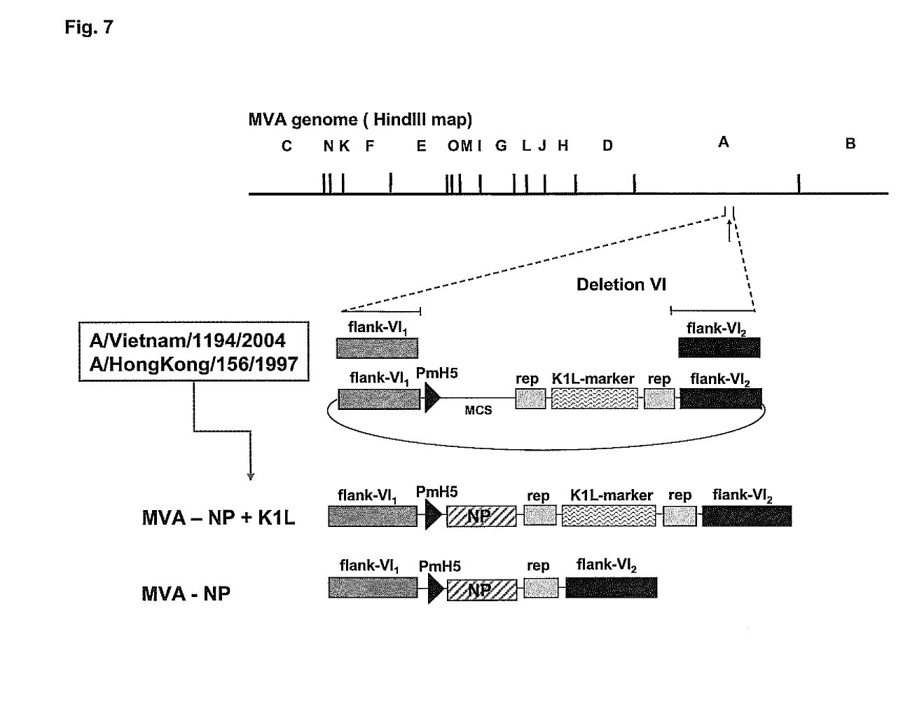

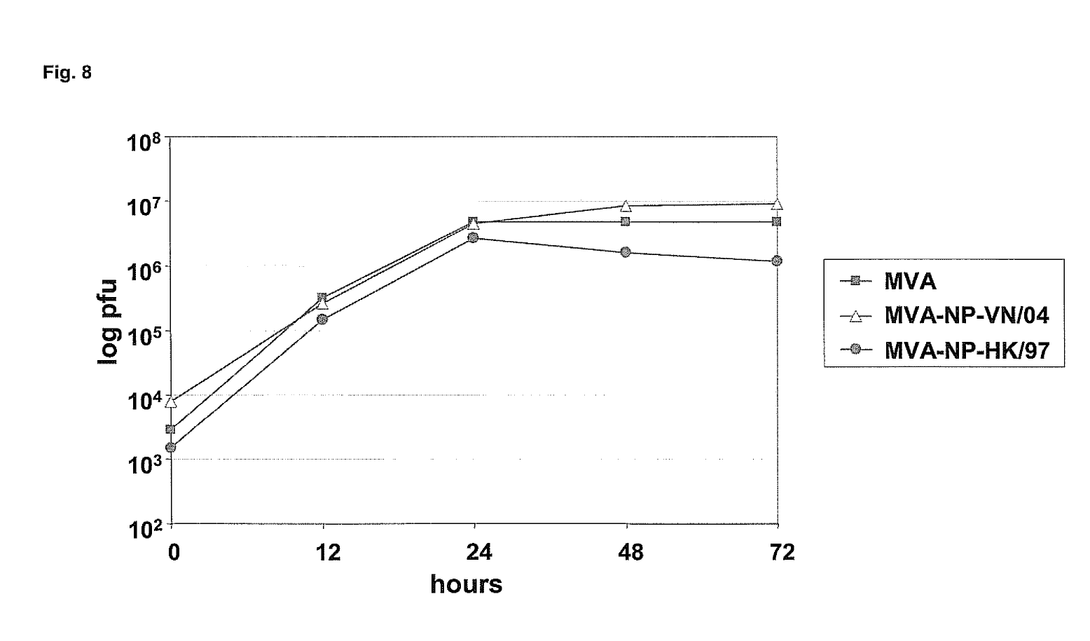

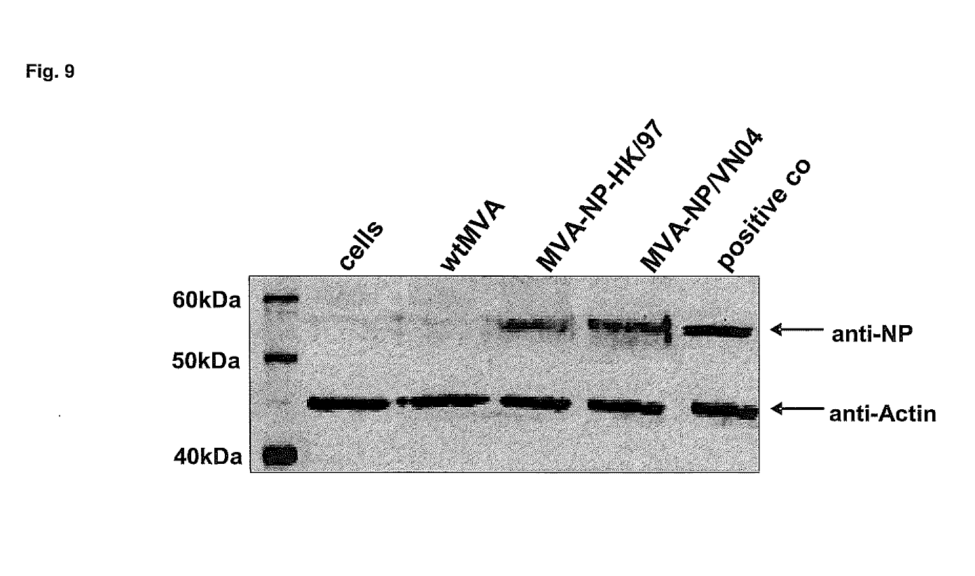

Antibody responses induced by vaccination. Antibody titers against the three challenge viruses: influenza virus A/Hongkong/156/07 ( [symbol] ), A/Vietnam/1194/04 ( [symbol] ) and A/Indonesia/5/05 ( [symbol] ) were measured by HI-assay (A+B). 28 days after the first immunization (A) and 28 days after the second immunization (B). Titers are presented as GMT (Log 2). Secondly antibody titers against the three different challenge viruses: influenza virus A/Hongkong/156/07 ( [symbol] ) A/Vietnam/1194/04 ( [symbol] ) and A/Indonesia/5/05 ( [symbol] ) were measured by VN-assay (C+D). 28 days after the first immunization (C) and second immunization (D). Titers are presented as GMT (Log 2).

FIG. 4

Histopathology and immunohistochemistry of the bronchioles and alveoli in lungs of mice infected with either influenza virus A/Hongkong/157/97, A/Vietnam/1194/04 or A/Indonesia/5/05 as indicated. Influenza virus A/Hongkong/156/97 infection leads to virus positive cells in the bronchiolar wall of wtMVA (A) and PBS immunized mice (E), combined with mild peribronchiolar infiltrate, while in the lungs of MVA-HA-HK/97 (B), MVA-HA-VN/04 (C) and Stimune®-adjuvanted NIBRG-14 (D) immunized mice no virus was detected. Infection with influenza virus A/Vietnam/1194/04 results in virus positive cells in the bronchiolar walls of wtMVA (F), MVA-HA-HK/97 (G) and PBS (J) immunized mice, also combined with moderate peribronchiolar infiltrate (except for the PBS immunized mice). No virus replication or morphological changes were detected in MVA-HA-VN/04 (H) and Stimune®-adjuvanted NIBRG-14 immunized mice (I). Inoculation of influenza virus A/Indonesia/5/05 resulted in severe infection of the bronchioles of wtMVA (K), MVA-HA-HK/97 (L) and PBS (O) immunized mice, combined with moderate peribronchiolar infiltrate. Whereas only a minimal amount of cells in the bronchiolar wall of MVA-HA-VN/04 (M) immunized mice was infected, combined with moderate infiltrate. No virus was detected in the lungs of Stimune®-adjuvanted NIBRG-14 (N) immunized mice after infection with influenza virus A/Indonesia/5/05.

FIG. 5

Construction of a Recombinant MVA According to the Invention

After introduction of the gene into the MVA transfer vector pIIIΔHR under the control of the vaccinia promoter the gene was inserted into deletion III of the MVA genome by means of homologous recombination of the flanking regions (flank 1 and flank 2). Selection of recombinant viruses was performed by expression of the host range gene K1L.

FIG. 6

Scheme of Construction for a Multivalent MVA-H5N1 Vaccine

The H5N1-antigen coding sequences were introduced into the MVA genome as follows: Matrix protein (M1)-, ion channel protein (M2)- and the neuraminidase (NA)-gene sequences of A/Vietnam/1194/2004 (H5N1 subtype) are targeted to the site of deletion II being placed under the transcriptional control of the vaccinia specific promoters P7.5, PmH5, P11. The nucleoprotein (NP) gene sequences of A/Vietnam/1194/2004 and A/HongKong/156/97 (both H5N1 subtypes) are controlled by PmH5 promoters and were introduced into deletion IV of the MVA genome (for details see FIG. 7). The hemagglutinin gene sequences of A/Vietnam/1194/2004 and A/HongKong/156/97 (both H5N1 subtypes) were inserted into the site of deletion III under control of vaccinia promoters PsynII.

FIG. 7

Construction of Two Different MVA-H5N1-NP Vaccines

Transfection of DNA from the MVA transfer vector plasmids pVIdHR-NP-VN04 or pVIdHR-NP-HK97 into MVA infected cells allowed the generation of the recombinant viruses MVA-NP-VN04 and MVA-NP-HK97. The NP genes are integrated in the MVA genome by means of homologous recombination into the site of deletion VI using the flanking regions (flank VI₁ and flank VI₂). Isolation of recombinant viruses was performed by growth selection using transient expression of the host range gene K1L.

FIG. 8

Multiple Step Growth Analysis of Recombinant MVA-H5N1-NP Viruses

Recombinant MVA carrying the NP genes from influenza virus A/Hongkong/156/97 (MVA-NP-HK97) or A/Vietnam/1194/04 (MVA-NP-VN04) were analysed for replicative capacity in cultures of chicken embryo fibroblast cells (CEF). Growth capacity and growth kinetics of both recombinant viruses were very comparable to those determined for non-recombinant MVA (wtMVA).

FIG. 9

Expression of NP-Antigens by MVA-H5N1-NP Constructs

The synthesis of influenza virus nucleoproteins was confirmed upon infection of CEF with MVA-NP-HK/97 and MVA-NP-VN04 recombinant viruses. Mock infected CEF and allantoic fluid containing A/PuertoRico/08/34 (subtype H1N1) served as controls. NP protein was detected using mouse monoclonal antibody HB-65 directed against the influenza virus nucleoprotein (ATCC-number HB-65).

1. A method using of a recombinant modified vaccinia virus Ankara (MVA) comprising a heterologous nucleic acid comprising producing a medicament in particular a vaccine, with said recombinant modified vaccinia virus Ankara (MVA) wherein the sequence of heterologous nucleic acid is from
influenza A virus class H5 antigen.

2. A method according to claim 1, wherein the heterologous nucleic acid is incorporated into a non-essential site within the genome of MVA.

3. A method according to claim 2, wherein the heterologous nucleic acid is incorporated into the MVA genome at the site of deletion III.

4. A method according to claim 1, wherein the heterologous nucleic acid is under the control of a vaccinia virus-, or orthopoxvirus-, or poxvirus-specific promoter.

5. A method according to claim 1, wherein the sequence of the heterologous nucleic acid is selected from the group of H5N1, H5N3, H5N2, H5N7, H7N1, H7N7, H7N3, and H9N2 sequences.

6. Use according to claim 5, wherein the sequence of the heterologous nucleic acid is from H5N1 and is selected from the group of strains termed, A/Vietnam/1203/04, A/Vietnam/1194/04, A/Vietnam/3046/04, A/Hongkong/156/97, A/HongKong/212/03, A/HongKong/213/03, A/Indonesia/5/05, A/Ck/Indonesia/BL03/03, A/Grey heron/HK/793.1/02, A/Black Headed Gull/HK/12.1/03, A/Dk/Vietnam/11/04, A/Ck/Vietnam/33/04, A/Ck/Vietnam/C-58/04, A/Dk/TH/D4AT/04, A/Gs/TH/G7CS/04, A/Dk/FJ/1734/05, A/Ck/GX/463/06, A/Ck/HK/947/06.

7. A method according to claim 6, wherein the sequence of the heterologous nucleic acid of H5N1 is that of strain A/Vietnam/1194/04, A/Indonesia/5/2005, A/Bar headed goose/Qinghai/1A/2005, A/Whooper swan/Mongolia/244/2005, A/turkey/Turkey/1/2005, and A/Anhui/1/2005.

8. A method according to claim 1, wherein the sequence of the heterologous nucleic acid encompasses the sequence or part of the sequence of the hemagglutinin gene from nucleotide 1 to 1500, from nucleotide 1 to 1100 or from nucleotide 40 to 1100.

9. A method according to claim 6, wherein the nucleic acid encompasses HA1 and HA2 subunits.

10. A method according to claim 6, wherein the nucleic acid encodes two or more sequences stemming from
a. different genes or parts thereof of a single H5N1 strain and/or,
b. different H5N1 strains.

11. A method according to claim 10, wherein the two or more sequences encode hemagglutinin or a part of hemagglutinin and stem from, A/Vietnam/1203/04, A/Vietnam/1194/04, Vietnam/3046/04, A/Bar headed goose/Qinghai/1A/2005, A/Whooper swan/Mongolia/244/2005, A/turkey/Turkey/1/2005, and A/Anhui/1/2005, and/or A/IND/5/05.

12. Recombinant modified vaccinia virus Ankara (MVA) comprising a heterologous nucleic acid, wherein
a. the heterologous nucleic acid is incorporated into a non-essential site within the genome of MVA,
b. the heterologous nucleic acid is under the control of a vaccinia virus promoter, or orthopoxvirus promoter, or poxvirus-specific promoter and,
c. the heterologous nucleic acid is selected from the group of nucleic acids encoding a gene or a part of a gene from an influenza A virus class H5.

13. Recombinant modified vaccinia virus Ankara (MVA) according to claim 12, wherein the heterologous nucleic acid is selected from the group of H5N1, H5N3, H5N2, H5N7, H7N1, H7N7, H7N3, and H9N2 genome sequences.

14. Recombinant modified vaccinia virus Ankara (MVA) according to claim 13, wherein the heterologous nucleic acid is from H5N1 and is selected from the group of strains termed, A/Vietnam/1203/04, A/Vietnam/1194/04, A/Vietnam/3046/04, A/Hongkong/156/97, A/HongKong/212/03, A/HongKong/213/03, A/Indonesia/5/05, A/Ck/Indonesia/BL03/03, A/Grey heron/HK/793.1/02, A/Black Headed Gull/HK/12.1/03, A/Dk/Vietnam/11/04, A/Ck/Vietnam/33/04, A/Ck/Vietnam/C-58/04, A/Dk/TH/D4AT/04, A/Gs/TH/G7CS/04, A/Dk/FJ/1734/05, A/Ck/GX/463/06, A/Ck/HK/947/06.

15. Recombinant modified vaccinia virus Ankara (MVA) according to claim 14, wherein the heterologous nucleic acid of H5N1 is selected from the strains A/Vietnam/1194/04, A/Indonesia/5/2005, A/Bar headed goose/Qinghai/1A/2005, A/Whooper swan/Mongolia/244/2005, A/turkey/Turkey/1/2005, and A/Anhui/1/2005.

16. Recombinant modified vaccinia virus Ankara (MVA) according to claim 15, wherein the sequence of the heterologous nucleic acid encompasses the sequence or part of the sequence of the hemagglutinin gene.

17. Recombinant modified vaccinia virus Ankara (MVA) according to claim 16, wherein the nucleic acid encompasses HA1 and HA2 subunits.

18. Recombinant modified vaccinia virus Ankara (MVA) according to claim 12, wherein the nucleic acid encodes two or more sequences stemming from
a. different genes or parts thereof of a single H5N1 strain and/or,
b. different H5N1 strains.

19. Recombinant modified vaccinia virus Ankara (MVA) according to claim 18, wherein the two or more sequences encode hemagglutinin or a part of hemagglutinin and are selected from sequences stemming from A/Vietnam/1203/04, A/Vietnam/1194/04, A/Vietnam/3046/04, A/Hongkong/156/97, A/HongKong/212/03, A/HongKong/213/03, A/Indonesia/5/05, A/Ck/Indonesia/BL03/03, A/Grey heron/HK/793.1/02, A/Black Headed Gull/HK/12.1/03, A/Dk/Vietnam/11/04, A/Ck/Vietnam/33/04, A/Ck/Vietnam/C-58/04, A/Dk/TH/D4AT/04, A/Gs/TH/G7CS/04, A/Dk/FJ/1734/05, A/Ck/GX/463/06 and A/Ck/HK/947/06.

20. Nucleic acid encoding a modified vaccinia virus Ankara (MVA) according to claim 12.

21. Method of introducing a recombinant MVA according to claim 16 into a target cell comprising infection of the target cell with a recombinant MVA according to claim 16.

22. Method for immunization of a living animal body including a human said method comprising administering to said living animal body including a human in need thereof a therapeutically effective amount of the MVA according to claim 16.

23. Method according to claim 22, wherein the immunizing may be both prophylactic and/or therapeutic.
